(19) United States
(12) Patent Application Publication
(10) Pub. No.: US 2024/0390023 A1
(43) Pub. Date: Nov. 28, 2024

(54) AESTHETIC TREATMENT SYSTEMS AND METHODS

(71) Applicant: Revelle Aesthetics, Inc., Mountain View, CA (US)

(72) Inventors: Jonathan Podmore, San Carlos, CA (US); Arthur Ferdinand, Gilroy, CA (US); Pablo Acosta, Newark, CA (US); Earl Bright, II, Sunnyvale, CA (US); Joshua Makower, Los Altos Hills, CA (US); Evan VandenBrink, San Mateo, CA (US); Amanda White, Mountain View, CA (US); John Hanley, Manhattan Beach, CA (US); Michael Schaller, Louisville, CO (US); Alicia Holler, San Francisco, CA (US); Sam Rhea Sarcia, Lakeside, CA (US); Alejandro Garcia-Rubio, Berkeley, CA (US)

(21) Appl. No.: 18/797,859

(22) Filed: Aug. 8, 2024

Related U.S. Application Data
(63) Continuation of application No. PCT/US23/12826, filed on Feb. 10, 2023.
(60) Provisional application No. 63/309,035, filed on Feb. 11, 2022.
(60) Provisional application No. 63/333,470, filed on Apr. 21, 2022.
(60) Provisional application No. 63/411,196, filed on Sep. 29, 2022.
(60) Provisional application No. 63/413,710, filed on Oct. 6, 2022.

(51) Int. Cl. A61B 17/32; A61B 17/00

(52) U.S. Cl. CPC A61B 17/320016; A61B 2017/00747; A61B 2017/00792; A61B 2017/320028; A61B 2217/005

(57) ABSTRACT

Systems and methods for aesthetic treatment of skin including an apparatus that applies or a method involving separating tissue to eliminate or reduce the appearance of unwanted features. In one approach, an interventional tool is placed between tissue layers to engage and treat connecting tissue layers. Focal fat tissue collection is conducted as needed independently or supplemental to the treatment of targeted septa.

### FIELD OF THE DISCLOSURE

The present disclosure generally relates to aesthetic treatment systems and methods.

### BACKGROUND OF THE DISCLOSURE

There is a continuing need for an effective approach to improving the appearance of the surface of skin. In one or more approaches, it is desirable to treat cellulite, also known as gynoid lipodystrophy, nodular liposclerosis, edematofibrosclerotic panniculopathy, panniculosis, adiposis edematosa, demopanniculosis deformans or status protrusus cutis. Additionally, there is a need for treatment devices for use in scar release, acne subcision, facial fold and/or facial lift procedures. Moreover, there is a need for proactive treatment modalities that prevent future or reoccurrence of skin discontinuities and which are easy and effective to use.

It has been reported that more than 85% of women have cellulite thus suggesting that cellulite is a physiologic rather than pathologic condition. The existence of fat in the reticular dermis alone is not thought to cause cellulite. Cellulite can be described as the herniation of subcutaneous fat within fibrous connective tissue that is expressed as dimpling of the skin. This fat loading can lead to stress on connective tissue located between fat lobules. Such dimpling is more common in women than men due to the orientation of subcutaneous fibrous structures defining chambers containing fat cells. In fact, it is this structure that is believed to cause the appearance of cellulite more than being overweight. Often, cellulite appears on the pelvic region including the buttocks, lower limbs and abdomen.

Subdermal fat layers below the epidermis are contained between dermal layers connected by septa which act as connective tissue between the dermal layers. In men, the septa are arranged more randomly and densely oriented in a more criss-crossed configuration while the septa in women are generally more parallel in arrangement. Also, men have thicker dermis and more angled septa relative to the skin surface whereas women have relatively thinner dermis which thins with age, and septa that are perpendicular to the skin surface. Moreover, women with cellulite have exhibited thickening of the septa in the regions of cellulite and tensioning of septa highlights cellulite. In women, fat storage in adipose tissue has a biological purpose in that it is maximized ensuring adequate caloric availability for pregnancy and lactation. An increase in fluid retention or proliferation of adipose tissue in such subdermal fat layers can further result in the appearance of cellulite where the septa is maintaining a first distance between dermal layers, thus creating dimples, whereas pockets between septa bulges. Over time, the septa may stretch, then eventually contract and harden thus retaining tissue layers at fixed distances, but pockets between such septa may be expanded thus adding to the appearance of cellulite.

Various approaches have been taken to treat or address cellulite. Early treatments involved attempts at increasing circulation and fat oxidation in areas exhibiting cellulite. Here, substances such as hyaluronic acid and aminophylline were injected in the target areas to reduce cellulite. Other approaches involved electroporating the target areas followed by the application of mesotherapy, or applying dermological creams or other supplements to cellulite. These approaches could be supplemented by massage or massage was used alone for the purpose of promoting increased fat reabsorption or drainage of fluids and toxins in the treated areas. Ultrasound has also been proposed to disrupt subcutaneous tissues and fat and has been used in combination with liposuction. Low acoustic pressure in combination with the infiltration of microbubbles has also been employed to reduce the appearance of cellulite, as has the use of other energies such as lasers and radio frequency. Such approaches have been characterized by limited or unpredictable results. More recently, the cutting of septa with blades or needles in the subdermal region has been employed. Prior approaches have been found to be labor intensive and very traumatic to the tissue leading to numerous skin entry sites, bleeding, bruising, tough tissue nodules, long, painful recoveries and inconsistent results.

There is also a significant need for devices to treat scars and acne, or for use in facial fold and/or facial lift procedures. In conventionally available procedures, treatment devices are required to be inserted within tissue close to the interventional site, and travel within tissue to the site results in cutting or disrupting non-target tissue. There is thus a need for a treatment device that can be placed into tissue at more cosmetically desirable locations where the treatment device includes a blade that is only exposed once it reaches the treatment tissue. In addition, there is benefits associated with using a single entry site to treat multiple target locations through the single entry site since safe and minimally traumatic navigation between entry and the targeted tissue is desirable.

Accordingly, there is a need for effective and efficient approaches to improving the aesthetic appearance of skin including treating, minimizing or eliminating trauma. These approaches should be associated with predictable results and be relatively easy to employ.

The present disclosure addresses these and other needs.

### SUMMARY OF THE DISCLOSURE

Briefly and in general terms, the present disclosure is directed towards aesthetic treatment systems and methods involving an apparatus that facilitates and methods involving, depending on the system used and force applied by the user, stretching, re-orienting, disrupting, cutting, slicing, and/or tearing tissue. In one aspect, the treatment approach involves a tissue cutting or slicing system. In another aspect, tissue cutting or slicing is combined with disruption and/or localized removal of fat.

In one embodiment, a cellulite treatment device is mounted at a distal end portion of a shaft and is sized and shaped to be advanced between tissue layers. In one particular aspect, fibrous septa that connect superior and inferior fascia plateaus within skin can be crossed with the treatment device using one or more of an array of tools to engage, and depending on the tool used and force applied by the user, stretch, re-orient, tear, disrupt, cut or slice septa. By doing so, the target subcutaneous connective tissue associated with the surface defect can be directly modified with minimal impact to surrounding blood vessels and lymphatic system and fat can be more evenly distributed and skin can assume a smoother appearance.

In various aspects, a handle assembly is provided to actuate a treatment device. In one or more approaches, the handle assembly includes an actuatable assembly that controls configuring a treatment device into one or more of home or sheath, open or hook, and active or cut configurations. The actuatable assembly can include one or more of a slider, an active or cut button and a return home button. Alternatively, the handle assembly includes structure that facilitates an automatic return to home upon completion of stretching, re-orienting, tearing, disrupting, cutting or slicing of septa or other tissues.

In one embodiment, the stretching, re-orienting, tearing, disrupting, cutting or slicing of septa is performed in combination with localized fat disruption or removal. In one approach, a disruption or removal device is embodied in a cannula including a deployable and adjustable basket configured to engage and remove fat tissue. The cannula can be attached to a vacuum pump that provides suction for drawing fat into the basket for removal from an interventional site.

In one or more aspects, a cellulite treatment system embodies a tool facilitating an ability to reach and treat all target cellulite appearance areas through a single skin entry on each side of the patient or a limited number of entries through the skin. In certain aspects, such tool is sized, shaped and configured (e.g. less than or equal to about three millimeters diameter, more preferably less than or equal to about 2 millimeters, and blunt dissection tip) to be placed within and advanced between tissue layers on its own and without assistance from external skin stabilizing structure, such as a suction device. Entry points through the skin such as high on the hip under where a bikini or underwear strap would be and along creases or transitions between buttocks and thighs (for example, gluteal crease) are employed. Identification and assessment of target septa is accomplished by pushing, pulling or otherwise tensioning septa in areas believed to be associated with the appearance of cellulite on the outside of skin. It has been recognized that septa causing a dimple or depression are located at various angles and locations relative to the dimple or depression observed on the skin and are not necessarily directly below such appearances of cellulite, and the treatment system and method is configured to identify the septa responsible for the appearance of cellulite that has been marked on the skin and target treatment on those septa and leave adjacent septa, blood vessels, etc. intact. Moreover, a range such as a small subset or a larger number of septa can be the structure causing a particular depression or dimple.

In one method, anesthetic is injected into the treatment site transcutaneously or subcutaneously, a cellulite treatment system is inserted subcutaneously across the treatment site and used to identify the septa responsible for a depression or dimple by pushing or pulling on various septa to cause a depression in the skin in the target area, and a cutting or slicing device or septa disruption structure is placed subcutaneously at the treatment site and employed to engage and cut or slice or break the septa tissue. In one particular aspect, the patient is directed to clench their buttocks and/or leg muscles to help facilitate identifying target areas and after septa treatment confirm release of septa that create dimples or depressions. Alternatively, the physician can press in a cranial to caudal direction on the skin above the treatment target or pull from below the treatment target. Remote imaging or ultrasonic or fluoroscopic energy can be employed to observe the procedure. A resizing or alternative configuration of the treatment structure can be employed to complete the treatment of a particular area. The treatment device is then repositioned to treat additional areas. The treatment device can be configured to treat a plurality of areas simultaneously or in succession without removing from the patient or a spot treatment approach can be taken. Additionally, through one or more entry points, various treatment trajectories are directed and in certain applications a steerable introducer is used to access treatment areas. Further, anti-inflammatory, collagenase, deoxycholic acid, salicylic acid, glycolic acid, hyaluronic acid, tranexamic acid or cellulite treatment medicants can be employed at the interventional site separately or directly by the interventional device or other procedural instrumentation. Aspects of the current invention include specific identification of the septa responsible for the cellulite appearance, severing or separation of those septa, confirmation intra-operatively that the separation of those septa was accomplished and the prevention of the re-appearance of the cellulite.

In various aspects, the treatment device can include one or more of cutting structure that cooperates with a septa or connective tissue hooking or engaging element to one or more hook or engage then cut, slice, tear or disrupt septa or connective tissue. One or more of the septa or connective tissue hooking element and the septa or connective tissue cutting element is convertible from a hooking configuration to a cutting configuration and from a cutting configuration to a hooking configuration or to a stored configuration. The treatment device can further include various approaches to blocking structure that selectively blocks one or more cutting elements. In another particular approach, the treatment device is embodied in an elongate member insertable through the skin capable of expanding at least one region from a smaller state to a wider state, and when in the wider state is configurable to both hook and cut, slice or disrupt target septa or connective tissue. In one specific approach, a treatment device is embodied in an elongate member that is configurable to present a hooked cutting structure for use in treating cellulite, scar or connective tissue existing in tissue planes.

The cellulite treatment system also involves in certain approaches, illumination such as a bright light configured at or emitted through a tip of treatment structure or placed along or at strategic locations along treatment structure for the purposes of tracking advancement of the tool to the treatment site and locating intra-dermal structures at the treatment site. In this way, direct observation of the treatment device by transillumination through the skin is provided and positioning, depth and performance thereof subcutaneously is readily available to an operator.

In one or more further aspects, the disclosed devices can be used in scar release, acne subcision, facial fold and/or facial lift procedures, or in any area where tissue disruption is needed in the subcutaneous space between tissue layers of the body. Transillumination can be used or specific devices or systems can lack transillumination structures or assemblies. In various approaches, a treatment device can be placed into tissue at more cosmetically desirable locations where the treatment device includes a blade that is only exposed once it reaches the treatment tissue. In addition, a single entry site can be used to treat multiple target locations through the single entry site since safe and minimally traumatic navigation between entry and the targeted tissue is possible.

These and other features of the disclosure will become apparent to those persons skilled in the art upon reading the details of the systems and methods as more fully described below.

### BRIEF DESCRIPTION OF THE DRAWINGS

FIGS. 1A and B are perspective views, depicting cellulite on a subject's skin and a plan for treating the cellulite.

FIG. 1C is a top view, depicting treatment of a subject lying on a treatment table.

FIG. 1D is a top view, depicting a cellulite treatment assembly and approach for treating cellulite.

FIGS. 1E-G are side views, depicting the complex anatomy of septa.

FIGS. 2A-G are partial cross-sectional views, depicting one embodiment of treating septa below a skin surface.

FIGS. 3A-C are perspective, side and cross-sectional views, depicting one preferred embodiment of a treatment system.

FIGS. 3D-H are perspective views, depicting alternative approaches to treatment devices.

FIGS. 3I-J are side views, depicting an approach to sealing device components.

FIGS. 4A-C are top views, depicting alternative approaches to treatment devices.

FIGS. 5A-C are top views, depicting further alternative approaches to treatment devices.

FIGS. 6A-D are top views, depicting yet further alternative approaches to treatment devices.

FIGS. 6E-F are top views, depicting a treatment device including a flexible blocker.

FIGS. 7A-B are top views, depicting further approaches to treatment devices.

FIGS. 8A-C are top views, depicting additional alternative approaches to treatment devices.

FIGS. 9A-B are perspective views, depicting further alternative approaches to treatment devices.

FIGS. 10A-D are perspective views, depicting yet further alternative approaches to treatment devices.

FIGS. 11A-G are perspective views, depicting approaches to a disrupting and removal assembly.

FIGS. 11H-I are top views, depicting use of the disrupting and removal assemblies of FIGS. 11A-G.

FIGS. 12A-D are various views, depicting an alternative approach to a handle assembly.

FIGS. 12E-H are various views, depicting another alternative approach to a handle assembly.

FIGS. 13A-C are side views, depicting another two-button approach to a handle assembly.

FIGS. 14A-C are cross-sectional views, depicting yet another alternative approach to a handle assembly.

FIGS. 14D-F are cross-sectional and side views, depicting components of the handle assembly of FIGS. 14A-C.

FIGS. 15A-H are cross-sectional views, depicting other embodiments of a handle assembly.

FIGS. 15I-N are cross-sectional views, depicting handle assemblies with an automatic home feature.

FIGS. 16A-H are side views, depicting various alternative approaches to distal ends of a handle assembly.

FIGS. 16I-L are top views, depicting treatment assemblies with tissue piercing structure.

FIG. 17 is a perspective view, depicting a light needle assembly.

FIGS. 18A-D are perspective and top views, depicting further alternative approaches to a treatment device with certain structures shown as transparent.

FIGS. 19A-D are perspective and top views, depicting yet further alternative approaches to a treatment device with certain structures shown as transparent.

FIGS. 20A-C are top views, depicting a treatment device embodying slicing structure with certain structures shown as transparent.

FIGS. 21A-C are side and front views, depicting approaches to treating a face.

FIGS. 22A-B are perspective views, depicting another approach to a treatment device.

FIGS. 22C-D are enlarged views, depicting the handle of the device of FIGS. 22A-B with certain structure shown transparent.

FIGS. 22E-F are enlarged views, depicting a distal end portion of the device of FIGS. 22A-B with certain structure shown transparent.

FIGS. 23A-J are perspective views, depicting yet further approaches to treatment devices with certain structures shown transparent.

FIGS. 24A-B are perspective views, depicting a treatment device including a rotating blade.

FIGS. 25A-B are perspective views, depicting another deployable treatment device with certain structures shown transparent.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

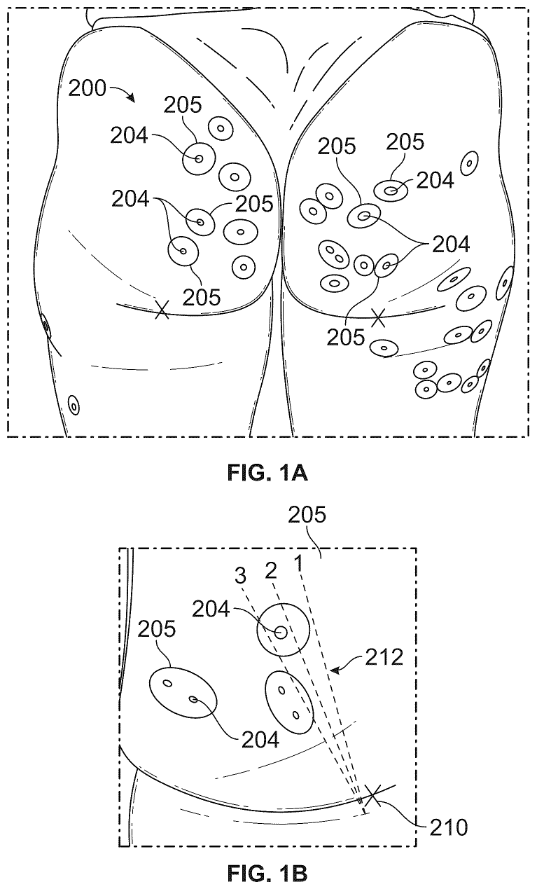

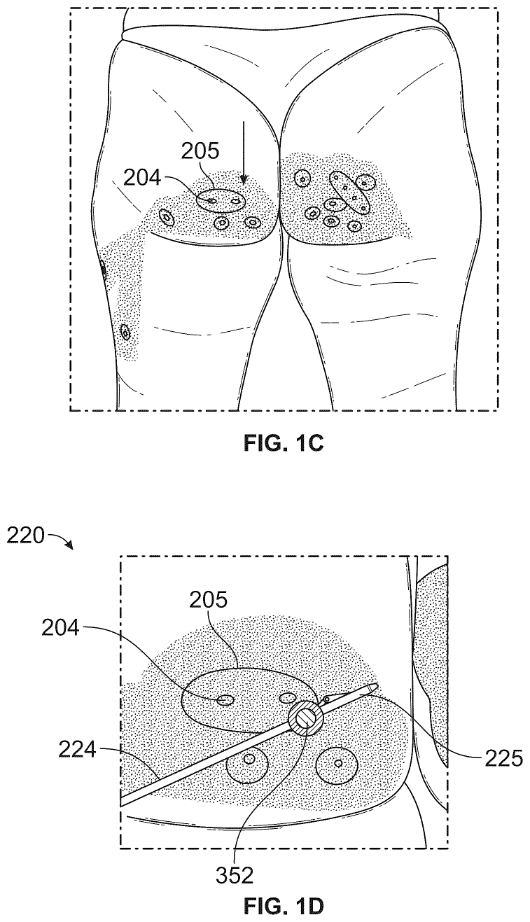

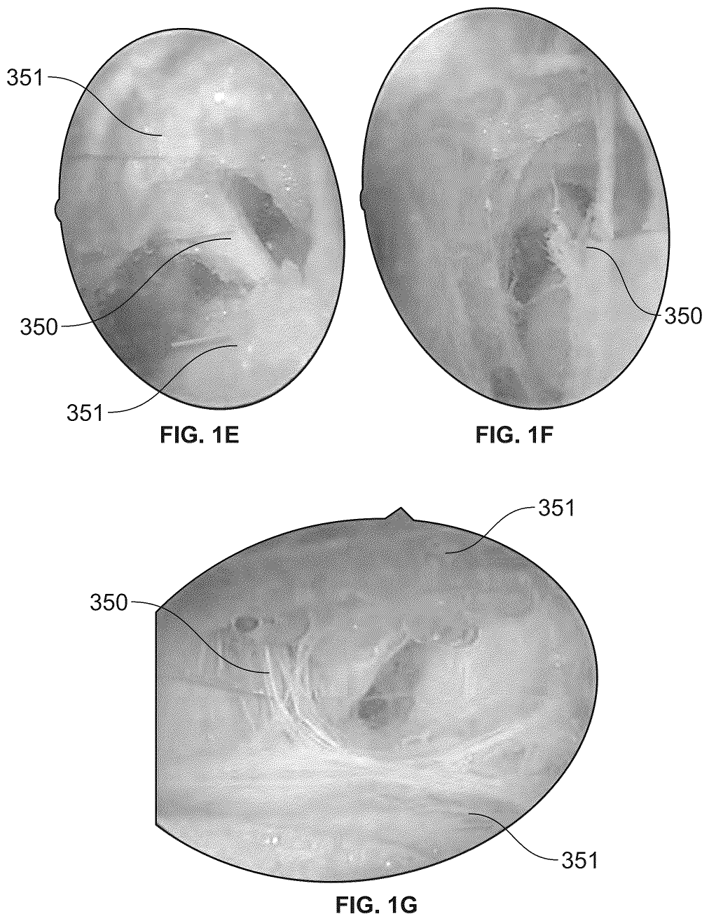

With reference to FIGS. 1A-B, there is shown a person exhibiting cellulite 200 about their thighs and buttocks. In one approach to treatment, dimples, linear depression and/or other depressions characteristic of the cellulite 200 intended to be treated are identified or circled with a marker to mark or outline edges 204 of depressions, preferably while the patient is standing as for most patients the appearance of their cellulite disappears when they lie down on their stomach because gravity is pulling in a different direction. The patient can be asked to lunge or clench tissue to aid in the identification of treatment areas. Approximately an 8 mm margin area 205 around targeted dimples, linear depressions or other depressions is marked on a patient's skin to identify an area for the physician to check for fibrous septa that are responsible for creating the dimple, liner depression or other depression. Such margins can assume various shapes dictated by the cellulite or depressions formed on a patient's anatomy and for example, can be defined as circles, ellipses or D-shaped treatment margins and the margins can encompass one or more targeted areas. In another embodiment, computerized imaging equipment is used to locate and mark dimples and/or depressions. In FIGS. 1A-B, dimples and depressions are marked for possible treatment. The physician treating the patient determines an instrument insertion site 210 and paths 212 that most efficiently treat cellulite with a minimal amount of insertion sites and instrument paths under the skin. Preferably, an instrument insertion site is chosen that is in a crease or fold of skin such as where the buttocks meets the thigh (i.e., gluteal crease) or in the crease between the two buttocks at a location that is not seen when the buttocks are in natural contact for improved cosmesis after the procedure healing period. Each depression can be divided into 6 mm lanes. With drawn 8 mm margins 205, each depression will thus have at least three lanes. Notably, multiple septa contribute to each depression. In certain patients, the inner thigh is chosen as an insertion site as this location is less visual as it heals, or the lateral thigh region or superior buttocks are used as alternative or additional insertion sites. Such treatment paths are selected by the operator preferably using a straight edge that bends or contours to the patient or can be generated automatically by employing a computerized controller programmed to most efficiently address and measure cellulite residing in a pre-defined treatment site. Each depression is treated from right to left since the treatment device deploys to the left and therefore treating from right to left makes it easier to keep track of which lanes have been treated. In various approaches, a treatment plan can involve treating sites in one or more of a counter-clockwise, right to left, lateral to medial or proximal to distal order. Once all lanes have been treated, an entire depression is verified from right to left and treated depressions are then marked with a surgical marker as being treated after verification. The computerized controller can be associated with a scanner that identifies specific dimples and areas for treatment such as by employing laser technology. In this regard, the computerized controller includes a program specific to cellulite treatment and is used in conjunction with an electronic and mechanical device and comprises or includes a non-transitory computer-readable storage medium and a computer-program mechanism embedded therein to both identify treatment areas and to plot primary and alternative approaches to treatments. In another embodiment, computerized visualization and treatment planning equipment is used to assist the physician in determining insertion site locations and paths to be taken to the marked targets.

Once a treatment approach is planned, the patient lies down on their stomach on the treatment table. Alternatively, because of the minimally invasiveness of the current approach, a patient can be treated while standing, particularly for a small number of treatment targets, or while standing and leaning forward on a support and alternatively between standing and leaning forward so that gravity can help identify and confirm treatment of the targeted septa. The patient can also be asked to lunge or clench muscles to aid in identifying treatment sites. Moreover, the measurement device creates a complete three-dimensional map of all cellulite relative to normal skin. By dating and comparing improvement of volume of divots or dimples versus normal idealized surfaces, the operator calculates total and local volume benefits of therapy and track improvement over time.

In one specific approach, as shown in FIG. 1C, anesthesia (represented by the shaded area) is thoroughly delivered subcutaneously beyond marked areas (approximately 1.5 inches) so as to provide room for a distal end of the treatment device 225 within the anesthetized area (see FIG. 1D). In one approach, multiple treatment targets along lines are treated from a single entry 210 at the lateral end of the gluteal crease (FIG. 1B). Thus, treatment can be directed to treat targeted septa, or additionally or alternatively, treatment can be transverse, preferably perpendicular, to the linear depressions on the posterior thigh or lateral thigh as shown in FIG. 1C for illustrative purposes to treat targeted septa. Treatment can be directed at various positions about connecting tissue or septa. That is, septa can be engaged, stretched, re-oriented, torn, cut, sliced, ruptured or disrupted from various sides or angles respecting septa. Thus, septa can be treated from above, below or the sides of septa to achieve the best results. For example, in a particular situation, treatment can be most effective from above a particular connecting tissue to take advantage of gravity where treatment forces placed on the connecting tissue coincide with the direction of gravity or the direction that gravity most often works on a standing body, as it has been observed that cellulite is often most visible in a standing individual. Additionally, using a limited thigh line treatment, the entirety of underlying septa associated with a linear depression in skin are not disrupted or treated but rather treatment involves approaching the linear depression perpendicular to or at an angle thereto and just a portion of the underlying septa is released. The treatment instrument is re-sheathed and repositioned slightly such as a few millimeters thereby leaving an area of undisrupted or treated septa, followed by disrupting or treating additional septa and subsequent re-sheathing, repositioning and disrupting or treating further septa as deemed necessary.

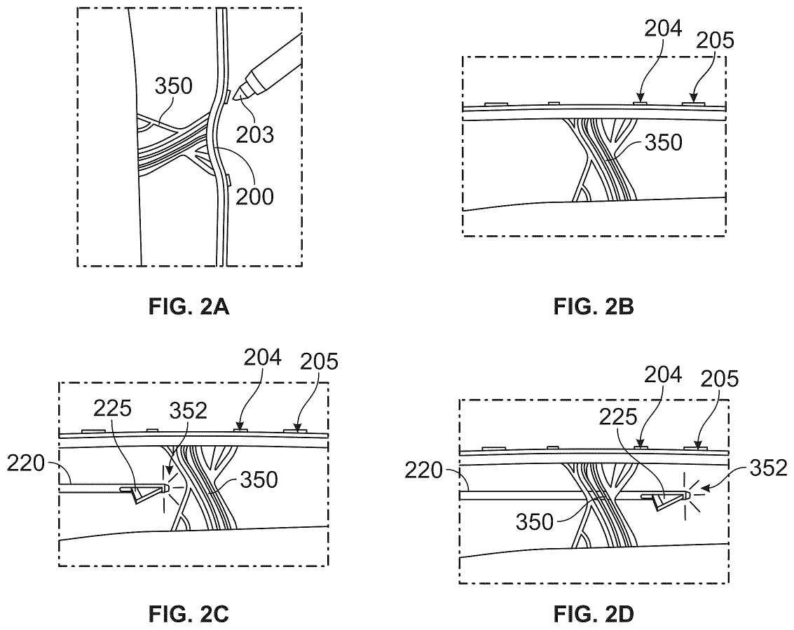

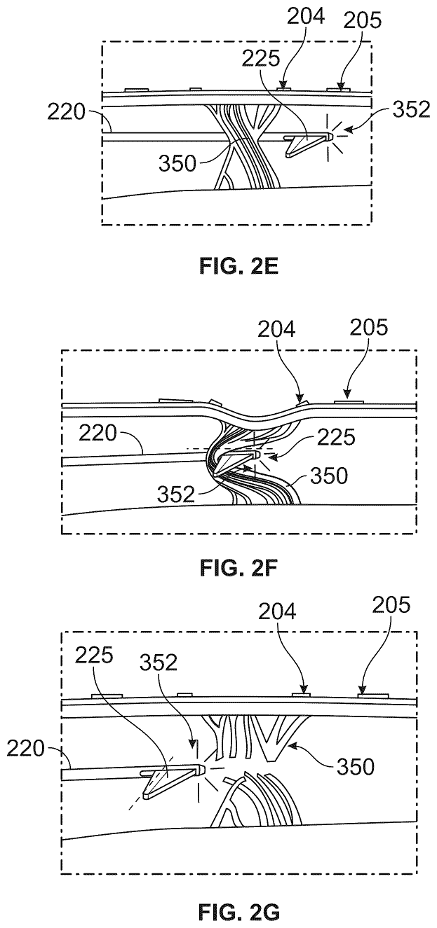

Turning now to FIG. 1D, there is shown a cellulite treatment assembly 220 and an elongate member or needle-sized structure preferably three millimeters or less, more preferably two millimeters or less, in diameter, like structure 224 extending longitudinally therefrom. A force gauge (electronic or mechanical) can be provided to ensure that a pre-determined amount of force would be applied to the tissue when testing the septa to prevent over or under pulling. A treatment device 225 capable of one or more of engaging, stretching, slicing, cutting or disrupting connective tissue is configured at a distal end portion of the elongate member 224 (e.g., FIGS. 2A-G). A light source 352 is provided proximal of the treatment device 225 to assist in guiding the treatment device 225 to targeted treatment locations. The treatment device can define an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure. All cutting means can be combined with or further energized with RF, a laser, ultrasonic or thermal energy to produce cutting and coagulation together or separately. Moreover, the cutting means can include a blade that is one or more of highly sharpened, hardened or coated (for example titanium nitride or Teflon).

In certain aspects, there can be a single entry site or two entry sites on each side of the patient, one high on the hip and another along the crease or transition between the buttocks and thigh, or at the inner thigh. Such locations are characterized in that they can be easily hidden either naturally or by clothing. Treatment targets, depressions and dimples that have been marked on the skin surface while the patient is standing often go away when the patient lies down on their stomach because gravity acts on the skin and underlying connective tissue in a different direction such that the ink mark 204 is apparent, but the dimple or depression is not. The disclosed interventional devices are configured such that a user can approach a target location and first use the interventional device to push, pull or otherwise tension septa in a target area under the skin to identify the specific septa impacting the target and/or which is the cause of the appearance of cellulite. In other words, pulling or pushing is performed on the septa under the skin to find the one(s) that create the dimple or depression in the skin surface. Notably, enough force is employed in pulling or pushing septa to create a dimple or depression on skin and an assessment is made to determine if the created dimple or depression corresponds to targeted dimples or depressions that have been marked for treatment. If so, then the engaged septa are treated as described herein and this approach is repeated for all targeted treatment areas. The operator also confirms that all the septa associated with a targeted dimple or depression have been treated with the treatment device so that all of the septa associated with targeted dimples or depressions have been completely released.

With reference to FIGS. 1E-G, it has been recognized that septa 350 define a complex network of connections between tissue layers 351 under the skin in and about the cellulite target areas, and septa includes “webs”, “trunks” and “branches from trunks” which connect tissue layers. It has also been recognized that septa can be quite elastic, stretching as much as about 10-20 mm before it creates depressions associated with the targeted area. In other circumstances, septa are inelastic and distances of about 10-25 mm are involved in creating enough tension for a dimple or depression to appear on the skin because of needed length of movement to tension long septa. Verification that all septa associated with a treatment target have been cut thus can involve multiple passes within a target area to ensure that an entire network of septa have been cut. Special attention is given to secondary septa that can be difficult to identify until after more readily identifiable or primary septa are cut. Secondary septa are septa that cause a shallower or smaller depression than the primary septa. Shallower or smaller depressions are not as noticeable on the skin when a patient is standing but after the primary septa to have been cut and there is no more deep depression, the shallower or smaller depression caused by the secondary septa becomes much more noticeable. Moreover, secondary septa and patterns of septa are expected and consequently searched in areas where there are multiple or closely located appearances of cellulite. In this way, a precise approach to relieving connective septa is taken in that septa hooked that create a depression outside the marked treatment area are released uncut and septa associated with targeted cellulite are cut. By taking multiple precision passes under a target area, all cellulite-forming septa, primary and secondary, are treated.

For some treatment targets, taking an approach from an entry located inferior the treatment target, advancing the end of the interventional device beyond the treatment target and then pulling inferiorly (effectively the “down” direction if the patient was standing) can provide a better approach, for example, for treatment targets on the leg, to re-create the dimple when the patient is lying down. One or more strain gauges can be incorporated within the treatment device to help identify target septa as well as to assess the progress and completion of treating septa. This facilitates targeting of key septa in a less impactful way, ideally minimizing bruising or other issues associated with cutting or disrupting a large area around the target. There are thus herein shown various approaches to treating cellulite expressed as dimples or depressions 200 in the skin surface. Moreover, the handle portion can be employed to create an indentation in skin through which interventional devices can be inserted subcutaneously. A treatment regimen is selected for inserting interventional instruments based upon the subject's anatomy as it relates to the septa 350 connecting tissue layers that define the chambers retaining fatty or other tissues. If desired, while anesthetic and/or sedation is taking effect, ultrasound can be used to assess the subcutaneous trajectory and depth of the various connective tissue bands responsible for the surface unevenness. The ultrasound evaluation can help with the particular trajectory selected for the desired depth. The ultrasound evaluation can also help with positioning the distal end portion of the treatment instrument strategically at the connection point between the connective tissue and the dermis or the facia.

As shown in FIG. 2A, targeted locations of cellulite to be treated can be marked 204 on the surface of the skin. This can be done when the patient is standing to best see cellulite. Cellulite can diminish or disappear when an individual is laying down, and should this happen, the marks 204 identify and confirm their locations (FIG. 2B).

In one aspect, local anesthetic is applied to the treatment site subcutaneously. In one approach, a long anesthesia needle (for example, 3.5 or 6 inches in length) is tunneled beyond a marked treatment site and anesthetic is administered under the marked area and along the tunneling pathway. It may be desirable to apply additional local anesthesia using a short needle transcutaneously to ensure that anesthetic extends beyond the marked target area. A distal end portion of a cellulite treatment assembly 220 is then inserted through the skin and the blunt tip is guided up into close proximity of the dermis as the tip can be tracked using the light source 352 at the distal end of the assembly as it is advanced toward septa 350 (FIG. 2C) near the marked location. Notably, the terminal end of the cellulite treatment assembly 220 in any of the disclosed embodiments can also define a tapered profile and include a tapered nosecone configured to assist in advancing the device between tissue layers. Entry sites are selected to both minimize post-operative healing time as well as to limit the use of anesthesia. The inventors have discovered given the goal of tensioning targeted septa 350, the distance from the marked location to where the treatment assembly 220 is inserted into the skin is preferably at least about 2 cm so that there is enough distance to pull and disrupt septa 350 and not have the tip of the cellulite treatment assembly exit the skin in the process. Additionally, a depth below the skin where septa 350 is preferably engaged (i.e., cut, sliced, torn, stretched, re-oriented (e.g. criss-crossing) or disrupted) is identified and determined. After determining the subcutaneous depth to be accessed for the cutting, slicing, tearing, stretching, re-orienting (e.g. criss-crossing) or disrupting of septum 350, the cellulite treatment assembly or other tool with a sharpened or blunt tip is inserted through the skin, advanced between subcutaneous tissue layers and toward septa 350. In one approach, a distal end portion of the cellulite treatment assembly is configured with a selectively activatable illuminated tip 352 that generates light (represented as illumination lines in the figures) with enough brightness to be seen through the skin. The intensity of light emitted by the tip 352 can be set to a specific constant level such that at the preferred depth below the skin for severing or otherwise engaging septa 350, the light that appears at the level of the skin as a circle or projection is of a pre-determined size. Thus, the treatment device is advanced to the target site. At the target site or as approaching the target site from the entry site, the user adjusts the depth of the tip of the treatment tool such that the circle or projection of light is the pre-determined size. Alternatively to having an illuminated tip, an illumination element can be located proximal of the treatment device 225. In this embodiment, the projection of light can be positioned under or alongside the target site such that the circle or projection of light is at the pre-determined location and the treatment element will be known to be beyond the pre-determined location. The septa 350 is tested and if confirmed as a target for treatment, the septa 350 is treated while maintaining the circle or projection at the pre-determined size. The user can also use the size of the circle or projection of light to maintain the depth of the tip of the treatment tool as it is advanced under the skin to the treatment target. In an alternative or another aspect, a sharpened tip is employed to create access to target tissue thus allowing the tool to create the desired path both into tissue as well as between tissue layers. It is expected that the depth that these tools are advanced will be between about 3 and about 10 mm below the skin surface, but it is anticipated that lesser and greater depths may also be optimal for a particular subject. It has been recognized that a more superficial treatment depth can be particularly effective in cutting all septa associated with a treatment site, as well as secondary septa are cut and can be cut in early passes within a treatment site. Accordingly, in a relative superficial approach the treatment device is advanced at a depth closer to the dermis than the superficial fascia. In any event, the depth selected is chosen for cutting, slicing, disrupting, tearing, stretching or re-orienting of the subject's septa 350. Moreover, in one embodiment, it is to be appreciated that the device 220 is formed from a substantially rigid material so that a consistent plane below the skin surface is accessed.

In another approach to precisely target and perform cellulite treatment procedures, a separate light source having a broad spread of light can be inserted into the target area through a separate incision or the same point that is less superficial to the skin layer. The treatment device is then advanced over this separate light source to identify where the treatment device is positioned through backlighting. By separating the light source from the treatment device, the treatment device can be less complicated, have fewer parts and constraints and can present a smaller profile requiring smaller incision sites and is easier to advance within tissue. Moreover, the treatment device would be subject to fewer electrical testing requirements and will potentially exhibit fewer electrical problems. In alternative or related approaches, a magnet can be incorporated into the treatment device and a magnet film can be placed over the skin to provide tracking of the treatment device. Also, a vein viewer-like apparatus can be employed to visualize reflectivity of a treatment device coated to demonstrate high reflectivity to identify device location, or near-IR light is employed to detect temperature differences between the treatment device and the patient's skin to thus identify treatment device positioning.

Using palpation, direct visualization (for example, transillumination or endoscopic) or non-invasive visualization (for example, ultrasound or fluoroscopic) or other means for determining the position of the interventional tool such as markings along the length of the instruments and its path within tissue, or providing the interventional instrumentation with radiopaque markers, the tool is placed at a site below where cellulite (for example a dimple) is seen on the subject's skin. The treatment device is advanced through septa 350 and to where the treatment device 225 is in a position best suited to accomplish the identification of target septa and the cellulite removal or minimization treatment. As shown in FIGS. 2D-E, in one approach, the treatment device 225 is passed beyond septa 350 in a stored or collapsed configuration, and once positioned beyond septa 350, a hook is deployed. Next, the tool assembly 220 is then pulled proximally to tension septa 350, such as by hooking the septa (FIG. 2F). In another approach, the treatment device 225 is passed a few millimeters lateral, preferably about 1 to about 10 millimeters, more preferably about 3 to about 6 millimeters, and beyond the target location, a hook is deployed and then swept laterally toward the target followed by pulling proximally to hook and tension septa. In one aspect, during initial deployment, the hook structure defines a relatively flat angle, that is, an edge of the hook is at about 80 degrees relative to a long axis of the elongate member 224, which results in providing the treatment instrument with substantial reach. Once it is time to cut or otherwise engage septa 350, the treatment device 225 is manipulated so that its blade or other cutting surface is exposed at a more steep angle better for cutting, such as about 70 degrees relative to the long axis of the elongate member 224. During these and other steps, transillumination can be employed to track the treatment device and guide the procedure. The marks 204 can facilitate targeting of septa 350 while using transillumination to see the location of the treatment device 225. In other approaches, a separate device can be employed to engage septa 350 to see if such septa are the source of a dimple or depression expressed on the outside of the skin. Such a secondary device can be placed remotely from the target (i.e. lesion) and configured to be capable of applying tension to the surface of skin in a predetermined direction so as to create the effect of gravity and produce the visualization of the lesions while the patient is in a prone position (i.e. a broad region of adhesive attached to a spring mechanism such that a predetermined force would be applied relatively parallel to the surface of the skin in the direction the skin would move when standing in gravity). Using this additional device could further help the confirmation and location of lesions and allow confirmation that the treatment was effective. Also, in various approaches, a portion of the elongate member can be configured to transition from a smaller state to a wider or larger state, wherein in the wider or larger state a cutting surface (i.e. sharpened edge or energy) is presented to cut tissue, the device being sized and shaped to be inserted through the skin and engage one or more regions of septa subcutaneously.

It is noted that septa causing a dimple or depression may be coming from various angles and locations relative to the dimple or depression seen on the skin rather than being directly below the dimple or depression, and may be due to one or only a few septa or a large number of septa that remotely cause the depression or dimple. Thus, so engaging certain septa will be reflected in some change in the dimple or depression on the skin. A determination is made concerning the correspondence with marks 204 made on the skin and the dimples being formed or re-formed. If the initial septa 350 that the user presses on or pulls on using the tool do not recreate a dimple or depression in the marked area 204, then the user releases those initial septa that were engaged and repositions the tool at different septa and presses on or pulls again. This is repeated until the septa responsible for a dimple or depression in the marked location are identified (FIG. 2F). Once proper septa are identified, the tool 225 is manipulated to cut, slice, disrupt, re-orient, stretch or tear septum 350 connecting tissue layers. In one approach, a blade 353 is deployed and presented for treatment.

After the proper septa have been severed, disrupted, stretched, or re-oriented, the treatment element 225 is moved back to its initial collapsed configuration. The treatment element is then advanced beyond the marked treatment location, the treatment element (e.g., hook) is deployed and then pulled back under the marked treatment location to confirm that all of the septa responsible for causing the marked dimple or depression have been separated intra-operatively. Again, multiple passes are taken to ensure that all cellulite-creating septa, including secondary septa are cut. If they have not been, the tool is manipulated to sever, disrupt, stretch, or re-orient additional septa. An approach is taken working right to left through an entire marked margin area and the steps are repeated until all of the septa responsible for creating the marked dimple or depression have been severed or sufficiently stretched and the dimple or depression cannot be re-created intra-operatively using the tool. Alternatively, or additionally, to check that the marked treatment targets have been adequately separated, treatment can be conducted with the patient lying down and the patient can then be asked to stand up off of the procedure table to let gravity act on the body to see whether marked treatment areas have been treated. Where the patient is asked to stand, steps are taken to maintain a sterile field and appropriate draping is provided to the patient. Where necessary, further treatment can be conducted on the unresolved areas. Such manipulation results in selective rupture, tearing, cutting or slicing of targeted septum 350, and the removal or minimization of dimples and the appearance of cellulite on skin (FIG. 2G). Thereafter, the treatment element (e.g., hook and/or blade) is retracted back in and the tool 220 is removed from the site to be withdrawn from the body or repositioned in any direction along and within the target tissue plane to treat additional areas.

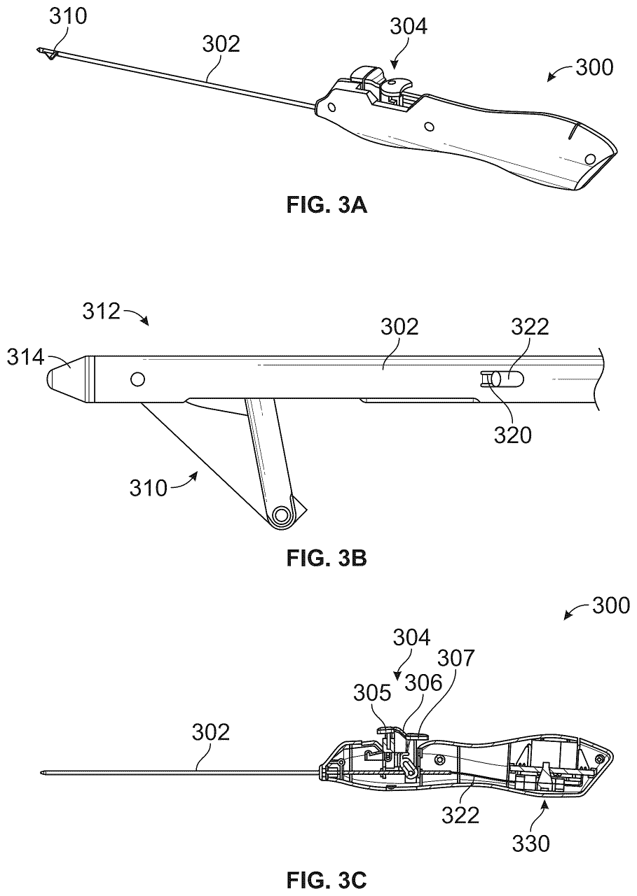

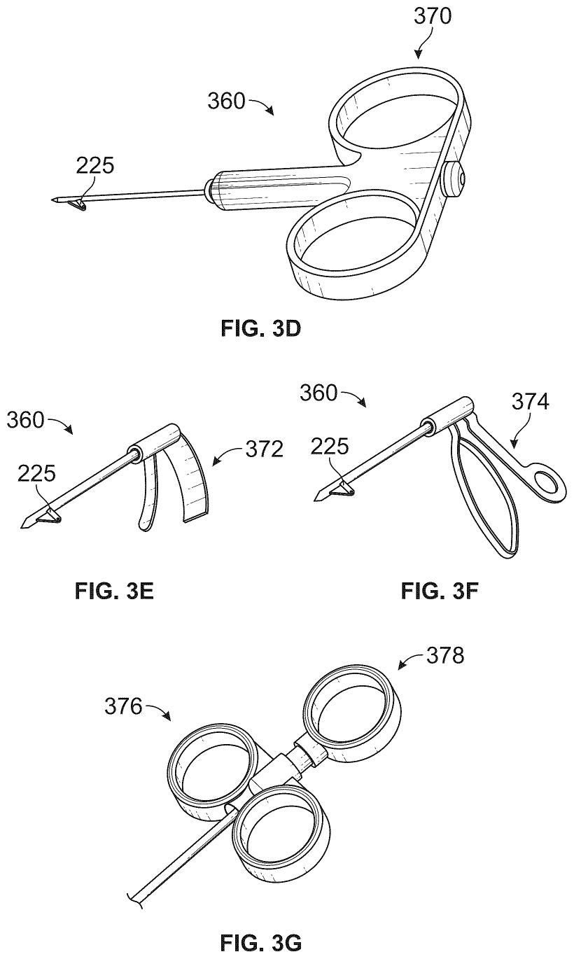

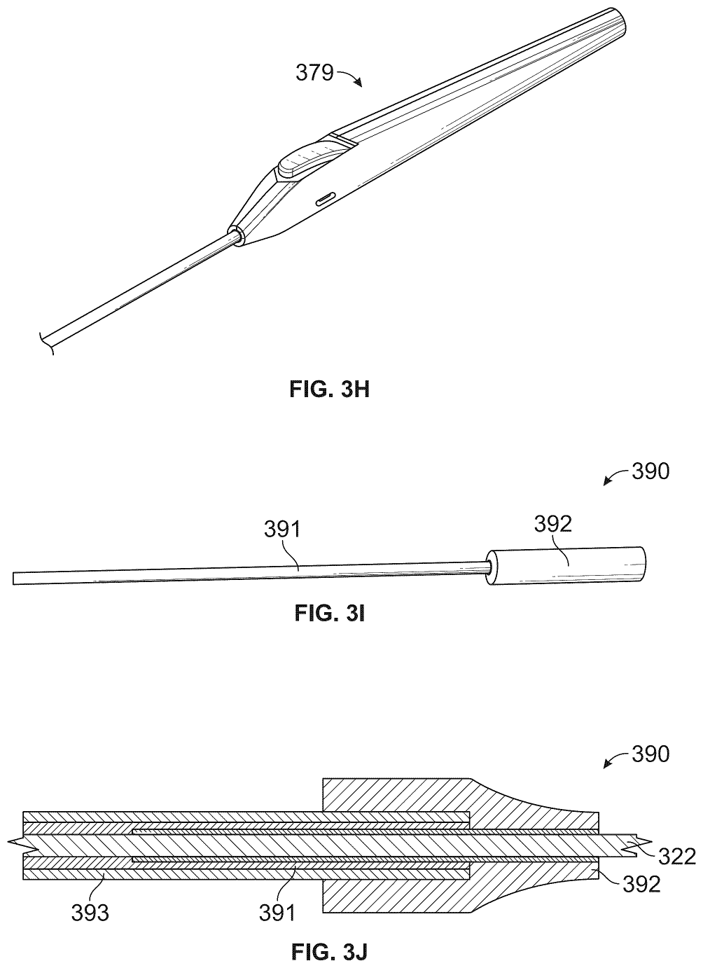

With reference now to FIGS. 3A-C, there is shown a treatment system that embodies certain functionality and a selected number of features described above. The treatment system includes a handle assembly 300 from which an elongate member 302 extends. The handle assembly 300 defines a contoured profile sized and shaped to conveniently fit within an operator's hand. Various actuating members 304 are provided on the handle assembly 300 the manipulation of which accomplishes the deployment of a treatment device 310 employed to engage, test and/or cut septa. The elongate member 302 has a length and cross-sectional profile configured to be placed between tissue layers and to be advanced and extend to septa existing in a target treatment area. A distal end 312 of the treatment system (FIG. 3B) includes the treatment device 310 and a terminal end 314.

The distal end 312 portion (FIG. 3B) of the treatment system is also equipped with an exit port 320 for a light fiber 322 that provides the transillumination functionality described above. As shown in the cross-sectional view of the treatment system (FIG. 3C), the light fiber 322 extends distally from a light energy source and focusing assembly 330 configured within the handle assembly 300. The light fiber 322 extends distally through the handle and within the elongate member 302 to the exit port 320. A battery provides energy to an LED which generates light energy.

In a complementary or alternative approach, touch-up or spot treatment of septa is possible employing a cellulite treatment system configured to address one interventional site at a time. Physicians or operators of such treatment devices or assemblies are thus provided with a tool to return to a patient to conduct follow-up treatments subsequent to a full or primary procedure. One of the disclosed deployable hook and cutting, treatment device assemblies 225 are configured at an end of the device in a simpler and lower cost assembly that does not contain all of the functionality of a primary treatment system. For example, such approaches can embody assemblies that omit a light source and assume an overall shorter or longer length than that of a primary treatment system. Various of the disclosed treatment devices can be included in such embodiments or alternatively, a treatment device can include a deployable blade without a blocking element. Here, the blade can be less sharp enabling the blade to both tension and check septa as well as cut septa when sufficient forces are applied. Moreover, such embodiments can additionally or alternatively include a static hook structure that is positionable to hook targeted septa. Accordingly, in certain approaches, cutting structures can be inserted perpendicular to skin to accomplish treatment or can be advanced below the skin in a direction generally parallel to the surface of the skin or angles with respect thereto.

In one or more approaches, a treatment system 360 includes a handle assembly that is provided for grasping by an operator to manipulate a treatment device 225 (FIGS. 3D-G). The handle assembly can one or more of a define a syringe handle arrangement 370 (FIG. 3D), a pistol grip actuation assembly 372 (FIG. 3E), a forcep actuation assembly 374 (FIG. 3F) or a control syringe handle assembly 376 that includes a hoop 378 for grasping by the operator (FIG. 3G). In yet a further approach, a handle assembly 379 can define a slim inline profile (FIG. 3H).

Extending longitudinally from one or more of the disclosed handle assemblies can be a needle assembly. The needle is configured to create an insertion site adjacent a specific cellulite target area, or directly into a dimple cellulite site. Further, it is through that interventional site instrumentation is advanced to address and treat septa residing below a dimple expression on a subject's skin.

As shown in FIGS. 3I-J, in certain embodiments, an approach to sealing fluid within a treatment device can be employed to inhibit fluid used or present during treatment procedures to be able to travel down a length of the device about an optical fiber 322, and impact device electronics. In one approach, an adapter insert 390 is configured to press fit into a proximal end of a pushrod of a treatment device. The insert 390 is comprised of two parts which press fit together. A first part 391 is a tube and the second part 392 is rubber tubing that wraps about the first part 391. The first and second parts 391, 392 can assume various dimensions and materials.

In one embodiment, the first part 391 is about 2.5 inches long and formed from 304 stainless steel, and the second part 392 is 15 mm long and formed from durometer Silicone rubber, about half of which wraps about the first part (FIG. 3J). This is done in such a way that the end of the first part is flush with the end of the second part, which itself grips the pushrod 393. The physical principle that allows for the restriction in flow rate is an adapted version of the Hagen-Poiseuille law which describes the pressure drop in an incompressible and Newtonian fluid in laminar flow. Though the Hagen-Poiseuille law generally applies to flow through a long cylindrical pipe of constant cross section, it can be adapted to annular flow by using a variable hydraulic diameter. The insert 390 is an example of annular flow control, that is, flow in which the law must be applied first to the flow between the optical fiber and the first part 391, and then between the first part and the pushrod 393. This geometry greatly increases fluidic resistance, and results in the flow rate to drop. Since the fluid path is driven by the equivalent of a constant pressure pump, increasing the length of the insert 391 decreases the flow rate. For an infinite length, it would take an infinite amount of work to push fluid through, and as such the flow rate approaches zero. For the treatment device, an insert with a length of 1.5-2.5 inches was sufficient to decrease the flow rate to acceptable levels (i.e. less than 3 drops/minute at 490 Pa pressure).

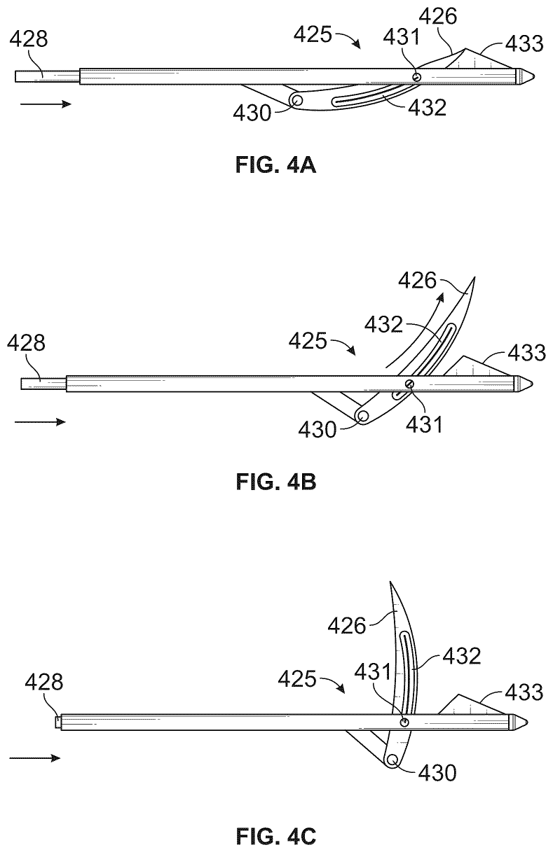

In one alternative approach to a treatment device (FIGS. 4A-C), there is provided a scythe assembly 425 defining a curved blade 426 that both rotates into position and penetrates tissue. The blade 426 is actuated by a pushrod 428 such that as the pushrod 428 is advanced, the blade 426 also advances into a target septa region. As the blade 426 is advanced it presents a hook configuration and also rotates about septa thus pulling targeted septa into the hook so assessment of the septa can be conducted. To accomplish this, in one approach, a curved slot 432 is provided in the blade 426 and the distal end portion of the treatment device is provided with a pin 431 about which the slot 432 is guided. The pushrod 428 can be retracted without cutting the captured septa or it can be manipulated to cut septa so that focal treatment of targeted septa can be accomplished. The distal end portion of the treatment device can also be equipped with generally triangular blocking projection 433 that covers a portion of the blade 426 when the assembly is in its stowed configuration for advancement or retraction within tissue. An additional blade blocking mechanism (not shown) can be added to the assembly and configured to cover the blade 426 during the hooking of selected septa, but allow for the penetration of a tip of the blade 426 into tissue.

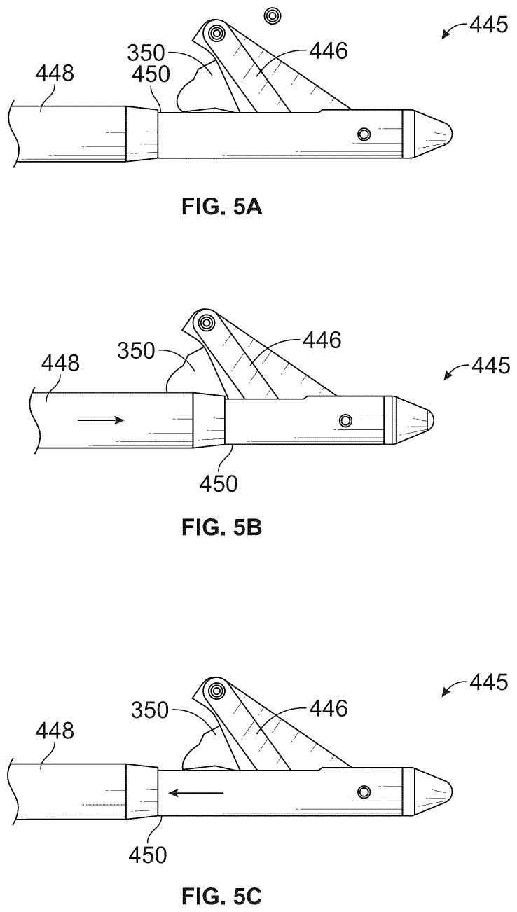

With reference to FIGS. 5A-C, there is shown a treatment device assembly 445 that includes a deployable hook and cutting assembly 446 in combination with a cylindrical reciprocating sleeve or hypotube 448 that includes a ground or sharpened distal end 450. This approach decreases the loads placed on a treatment device during cutting of septa. In use, the hook and cutting assembly 446 would capture targeted septa 350 and the reciprocating sleeve 448 would be employed to cut septa 350 and also advance septa 350 towards the static blade of the hook and cutting assembly 446 to be cut by the static blade. It is also contemplated that the assembly omit the static blade and cutting a septa captured by the hook assembly 446 is accomplished through the action of the reciprocating sleeve 448 alone. The reciprocating sleeve 448 can be one or more of longitudinally translatable and axially rotatable, and can be manually mechanically driven or driven by motors. A tissue ejection assembly involving a spring-loaded ejector (not shown) can be further incorporated into the assembly so that once tissue is severed, tissue is ejected as the blade is retracted. Also, a system of suction can be provided to draw cut tissue within a reservoir removed from the cutting action. In yet another approach, a rotating blade can be equipped with an ejector projection that encourages and lifts cut material away from the tissue cutting region of the device, allowing a next portion of targeted tissue to enter the cutting region.

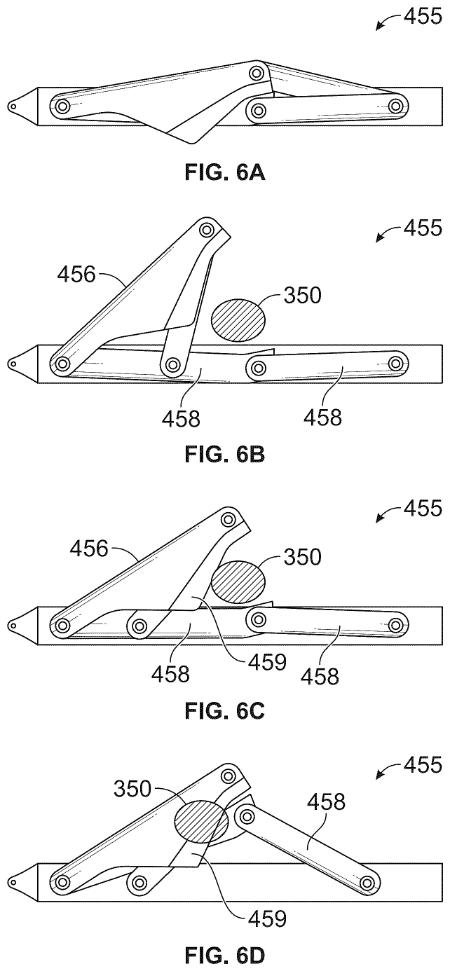

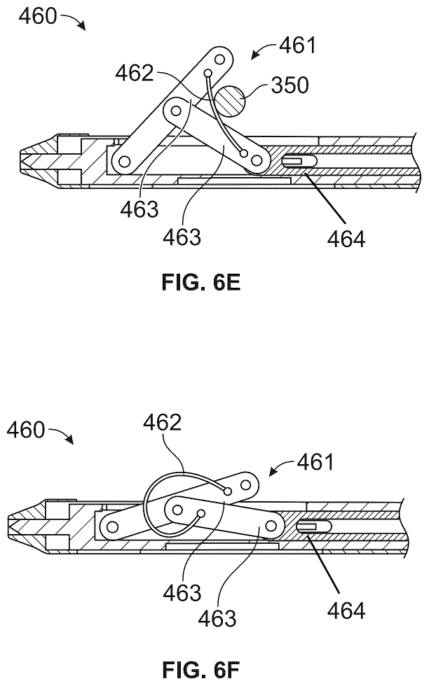

Turning to FIGS. 6A-D, there is shown a treatment device 455 characterized by an actuated cut functionality that provides more than a drag cutting approach to severing septa which can rely on forces higher than that needed to assess and/or tension targeted septa that has been hooked. An actuated cut allows for septa once identified and held, to be cut without any additional load being applied other than that of mechanism actuation and as such can reduce operator fatigue. This approach also lends itself to more precise treatments as unnecessary additional pulling of anatomy or septa outside of a target anesthetized zone can be avoided should a drag cut only be employed. The treatment device 455 also more easily disengages from septa that has been engaged and assessed. Accordingly, the treatment device 455 includes a hook and cutting subassembly 456 that cooperates with a pair of projectable links 458. In use, after the treatment device 455 is advanced to a target location in a fully stowed configuration (FIG. 6A), the hook and cutting subassembly 456 is deployed to capture and assess targeted septa 350 (FIG. 6B). Next, should it be determined that the captured septa should be severed, a cutting blade 459 of the hook and cutting subassembly 456 is exposed to the targeted septa 350 (FIG. 6C) and the links 458 are projected laterally to cooperate with the cutting blade 459 to sever septa 350 (FIG. 6D). Here, it is contemplated that at least a most distally configured link 458 can include a sharped blade to facilitate severing of septa 350 and the link 458 and blade 459 can be configured to slide over each other like a scissor or meet at their bladed edges. Various controls and set displacements of the links 458 can additionally be incorporated into the device so that desired positioning of the various cooperating components can be provided. The treatment device 455 can then be collapsed and stowed for advancement to another target site.

Referring to FIGS. 6E-F, there is shown a treatment device 460 including scissors 461 arranged to cooperate with a flexible blocker 462. Employing scissor structure lends itself to a device that accomplishes cutting of septa 350 while minimizing forces necessary to do so, and also presents traditional instrumentation for cutting tissue. The flexible blocker 462 is attached to blades 463 of the scissor structure 461. As shown, the flexible blocker 462 is defined by a wire but other structures such as bands or the like can also be utilized. A pushrod 464 operatively associated with the scissors 461 is provided to be manipulated by an operator to alternatively place the treatment device 460 is an open state (FIG. 6E) and an active state (FIG. 6F). When the treatment device 460 is in the open state by advancing the pushrod 464 forwardly, the blocker 462 is stretched or flexed with some amount of slack to form an arc, thereby blocking the blades or another cutting element 463 of the scissors 461. By withdrawing the pushrod 464 proximally, the treatment device 460 is placed in the active state and the blocker 462 gives way to the blades 463 thus allowing the device to engage and cut septa 350. Completely withdrawing the pushrod 464 places the treatment device 460 in a stowed position (not shown).

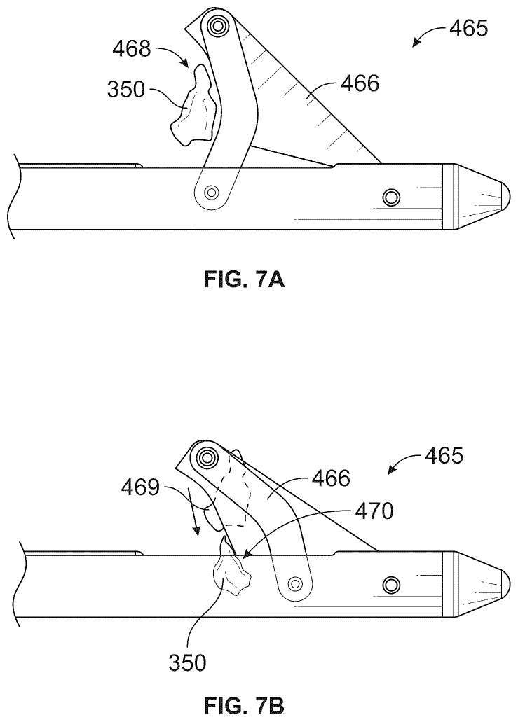

In yet other approaches to a treatment device, there are provided assemblies that accomplish septa severing through a sliding action (See FIGS. 7A-B and 8A-C). Generally, a more acute angle is presented to septa that drives tissue into a nook under tension and thereby provides relative motion between cutting surfaces and tissue to create a slice action. In one aspect (FIGS. 7A-B), the treatment device 465 includes a hook and cutting subassembly 466 that at one degree of lateral projection of the treatment device 465, the components define a more acute angle to present a hook nook 468 to capture septa 350. As the hook and cutting subassembly is closed proximally about captured septa 350, the septa 350 is pushed or forced along the blade edge 469 to a cut nook 470 where the severing of the septa 350 is completed. Relative motion between the blade edge 469 and septa 350 facilitates severing the septa using less forces. In another approach, a portion of an outer tube of a treatment assembly can be configured to flex outwardly so as to push tensioned septa along an exposed blade. In a related approach, when the treatment device is in a cutting configuration the blocker structure is moved further distally from a cutting blade edge which results in the treatment device requiring a lower force to cut target tissue, thus likely reducing an effect of having the blocker closer to the cutting edge. This moves cutting performance closer to a blocker-less blade for the purpose of ideally reducing necessary cutting force.

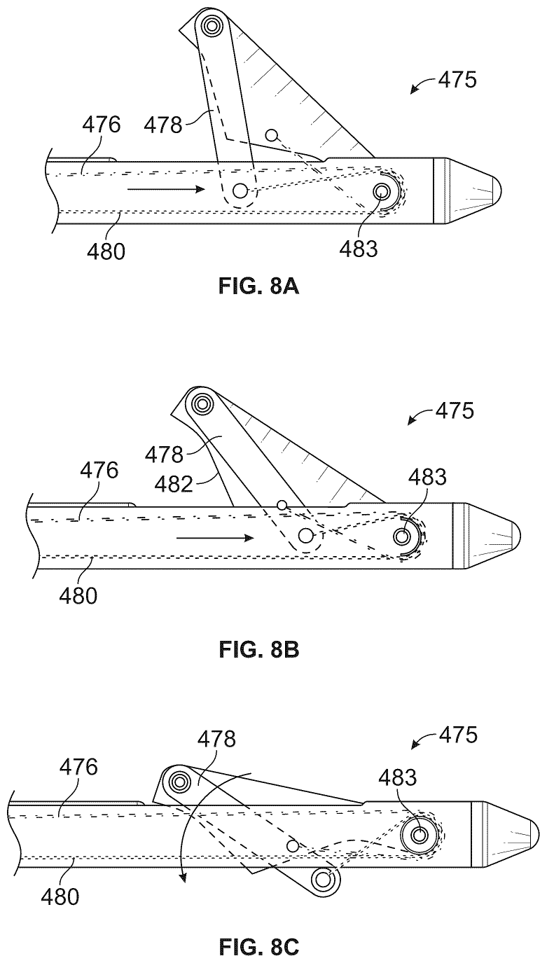

As shown in FIGS. 8A-C, in another aspect, a hook and cutting subassembly 475 is configured to move relative to tissue. Here, tensioned wire actuation is employed to control blocking and cutting structures. A blocker wire 476 is configured to place a blocker 478 as desired and a blade wire 480 is configured to position a blade 482 relative to a targeted septa 350. The blade wire 480 is routed about a pin 483 that holds the blade 480 and functions to push the blade 480 to create a sliding or slicing action against targeted tissue, the pin 483 acting as a pulley point. The pin 483 thus allows a length of a blocker wire 476 to remain the same during such cutting action as the hook and cutting subassembly 475 pivots with the blade wire tension. Accordingly, as tension is applied to one or more of the blocker and blade wires 476, 480, the hook and cutting subassembly 475 begins to close about targeted tissue and to provide a moving slicing action upon the tissue (FIG. 8B). Applying further tension to one or more of the wires 476, 480, further collapses the hook and cutting subassembly 475 (FIG. 8C).

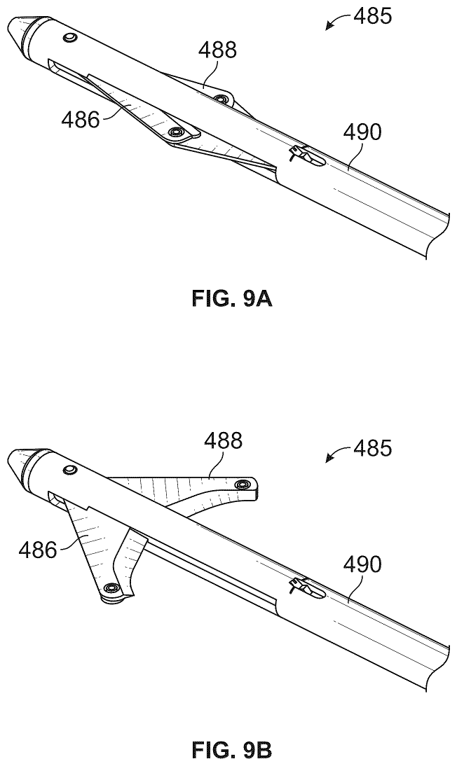

Referring to FIGS. 9A-B, a treatment device 485 can include two sets of deployable hook linkages 486, 488 connected to a single shaft 490 and projecting at opposite sides of the shaft 490 (See FIG. 9B). The deployable structure can be any one of the treatment device structures disclosed herein. By doubling the amount of tissue that can be captured and disrupted at one time, the number of cuts that an operator or physician has to make in one procedure to achieve desired results is reduced, potentially by half. Consequently, procedure time and by extension procedure cost is reduced.

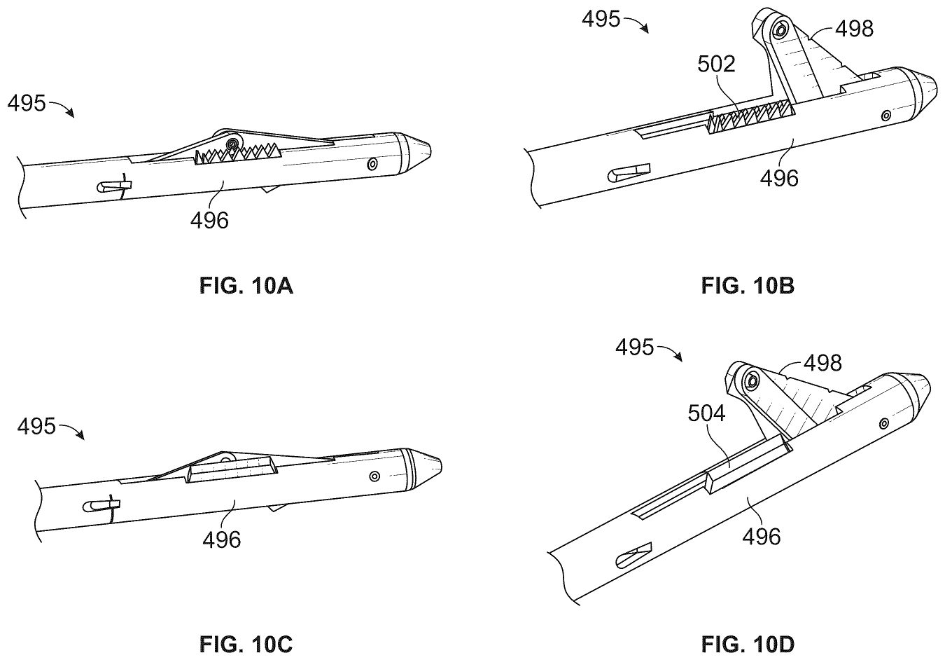

In another aspect, a treatment device 495 can embody a reciprocating blade 496 configuration in combination with a hook and cutting subassembly 498 (FIGS. 10A-D). This approach is characterized by cutting functionality that can be more thorough and complete with reduced effort on the part of the physician or operator, thereby shortening the procedure and reducing user fatigue. In one approach, the reciprocating blade is serrated 502 (FIGS. 10A-B) and in another, the reciprocating blade includes a straight edge 504 (FIGS. 10C-D). The serrated blade approach can resemble that of electric hair clippers or a hedge trimmer, with one stationary toothed blade juxtaposed with a similar reciprocating blade. Such blades can be driven by a battery powered electric motor located in the treatment system handle, with motion energy transmitted through a device shaft to the reciprocating blades 496. The hook and cutting subassembly 498 can be any of the approaches disclosed herein and as shown is positioned along the treatment device 495 distal to the reciprocating structure 496, and further, can lack a blade portion only using the hook to capture target tissue. In use, the hook and cutting subassembly 498 is employed to capture target tissue and after concluding such tissue should be cut, the hook and cutting subassembly 498 is manipulated to bring the targeted tissue into contact with one or both of the static blade of the hook and cutting subassembly 498 and the reciprocating blade structure 496.

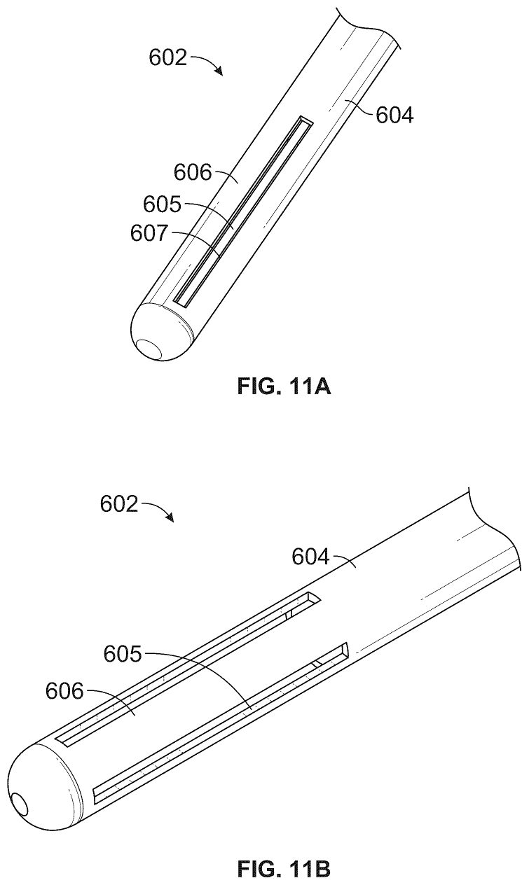

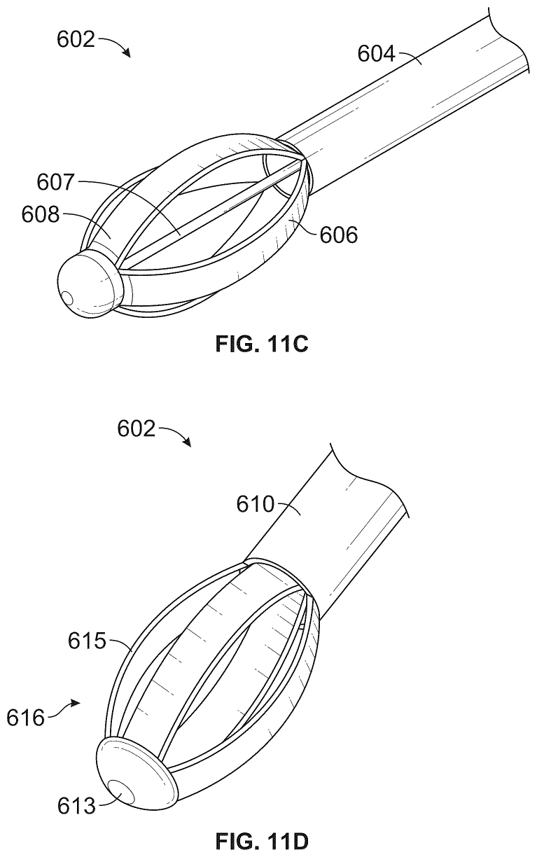

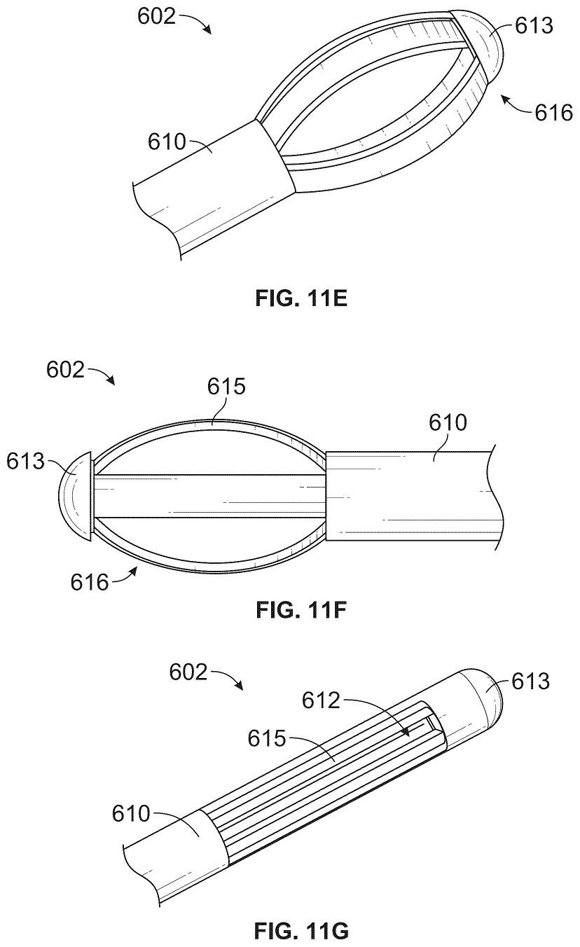

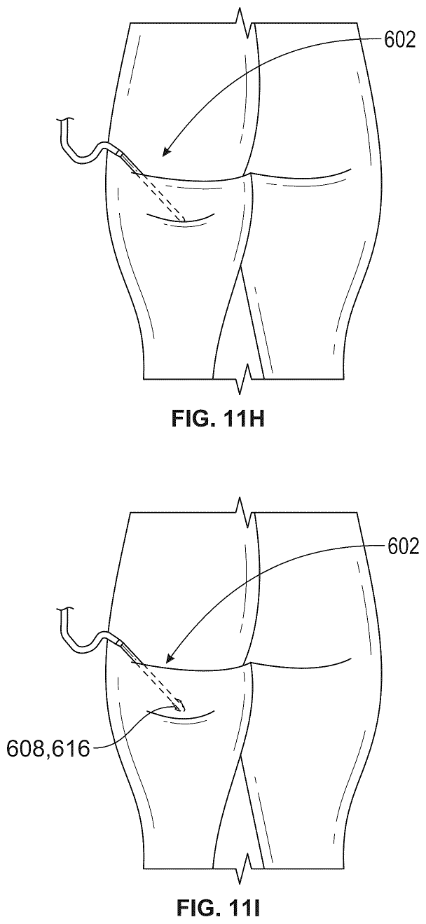

Turning now to FIGS. 11A-I, there is presented an elongate cannula assembly 602 configured to focally disrupt and remove fat from an interventional site. Such structure can be used independently or as part of an aesthetic treatment procedure involving stretching, re-orienting, tearing, disrupting, cutting or slicing of septa in connection with treating cellulite. It may be desirable where there has been septal release that local fat tissue collection be conducted and to take advantage of the procedural paths taken during septal release. As shown in FIGS. 11A-G, a collapsible and expandable basket assembly 608, 616 is attached at a distal end of an elongate shaft which can be one or more tubes that allow for the deployment, manipulation and size adjustment of basket structure. In another embodiment, the cannula assembly 602 is also adapted to be attached to a vacuum pump (not shown) for the purpose of collecting and removing fat from an interventional site. The assembly can be formed from plastic, stainless steel or nitinol tubes capable of allowing required outward deflection.

In one approach to a cannula assembly 602 (FIGS. 11A-C), the cannula assembly 602 is defined by a single tube 604 having a series of slots 605 configured in a distal end portion of the tube 602 to define flex members 606. A core shaft 607 is configured within the single tube 604 and connected to a distal end of the tube 602. Pulling the core shaft 607 controls the radial expansion of the flex members 606 at the distal end of the tube 604 as permitted by the geometry of the slots 605 to define a cage 608 for collection of fat tissue. In another approach to a cannula assembly 602 (FIGS. 11D-G), the device includes two concentric tubes that are joined together at a proximal end portion with a threaded connection (not shown). Other arrangements are also contemplated here such as a linear slide arrangement or an actuatable mechanism that imparts the desired relative motion. An outer tube 610 is configured to be rotated about an inner tube 612 and as it is rotated, a tip 613 of the cannula assembly 602 is drawn proximally. In doing so, flex members 615 configured at the distal end portion of the device are deflected outwardly to an amount dependent on the relative motion of the inner and outer tubes 610, 612 to thereby create a basket or cage 616 for the collection of fat tissue. Thus, the size of the cage 608, 616 can be adjusted by an operator or clinician to optimize minimal or precise sizing for target fat removal. In use, the cannula assembly 602 is inserted between tissue layers in its retracted state and once positioned as desired, the assembly is manipulated to present a basket 608, 616 configured for the focal collection of targeted fat tissue (See FIGS. 11H-I).

Various further alternative approaches to cellulite treatment systems can be employed. With reference again to FIGS. 3B-C, the actuating members 304 are provided on the handle assembly 300 the manipulation of which accomplishes the deployment of a treatment device 310 employed to engage, test and/or cut septa. Thus, manipulating the actuating members 304 results in the treatment device 310 assuming home or sheath, open or hook and active or cut configurations. In use, the actuating members 304 are in a first proximal position and the treatment device 310 is in the home or sheath position (See also FIG. 2C). Next, the actuating members are advanced distally by pushing on the slider 306 so that the treatment device 310 assumes an open or hook configuration (FIG. 2E) and thereafter, an active button 307 of the actuating members is depressed so that the treatment device defines an active or cut configuration (FIG. 2F-G). To return to home, the home button 305 of the actuating members 304 is manipulated so that the treatment device 310 returns to the home or sheath configuration. As described below, in further alternative approaches, the treatment system includes a handle that has fewer actuating members that are designed to facilitate the return of the treatment device to a home or sheath configuration directly from an active or cut configuration.

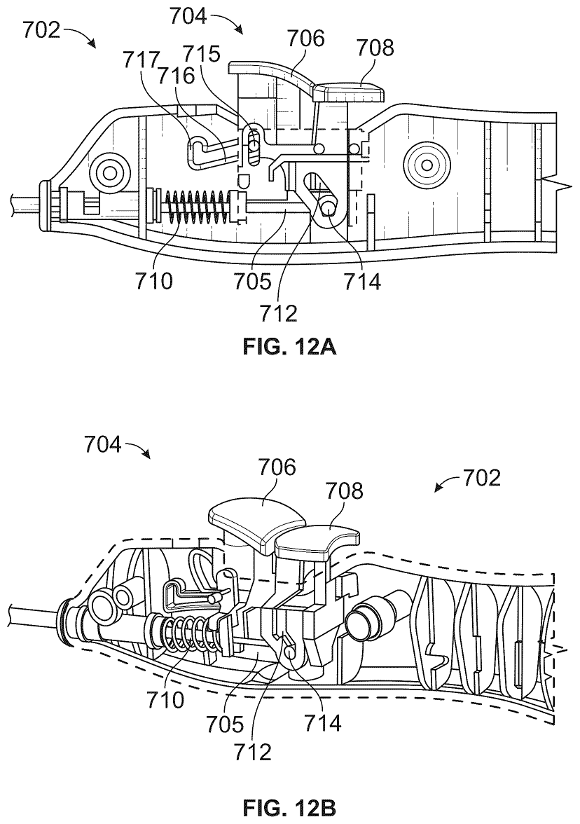

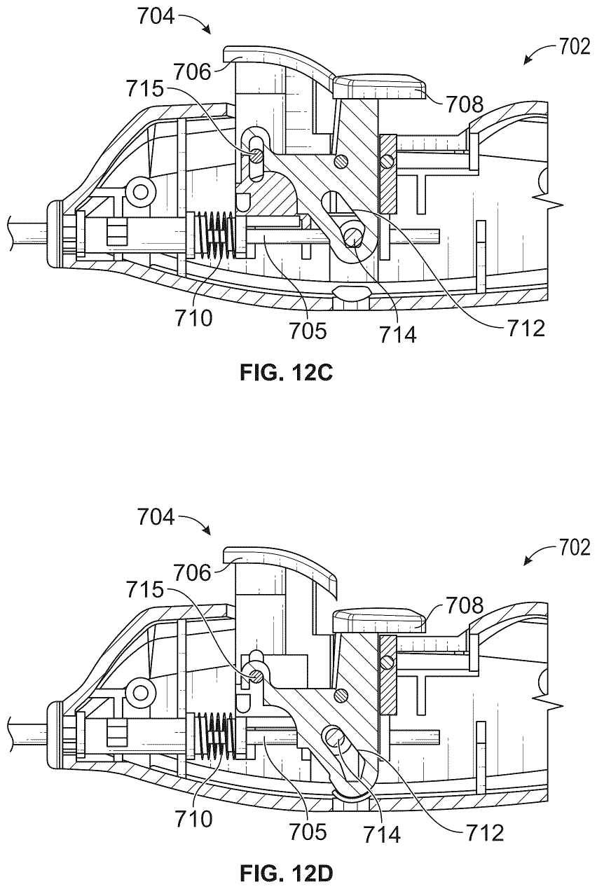

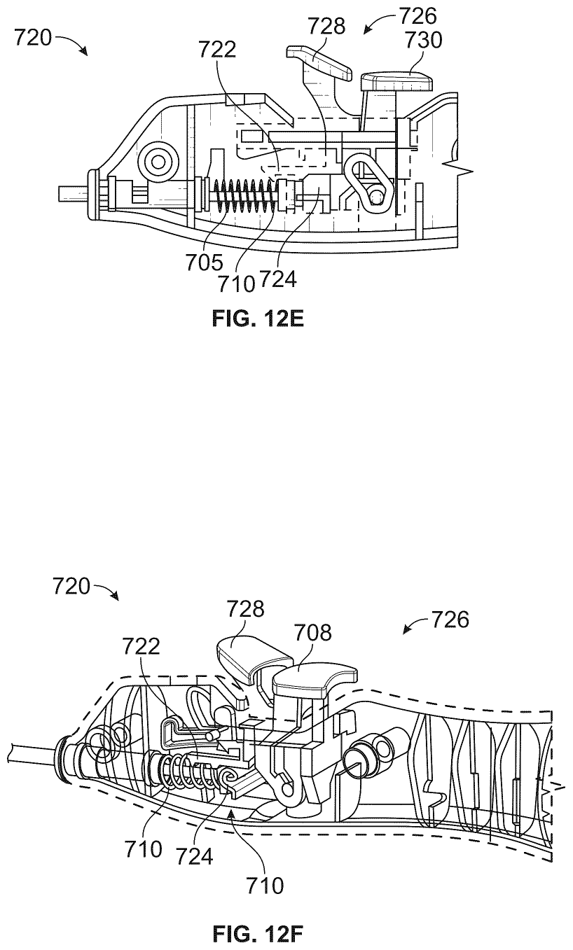

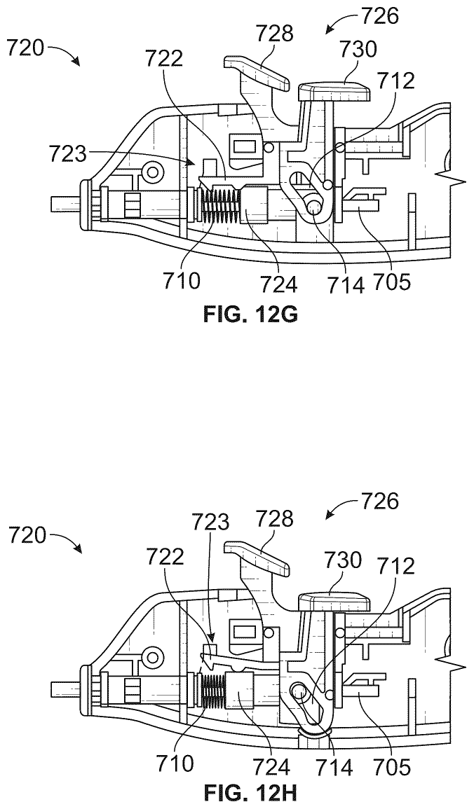

With reference to FIGS. 12A-D, there is shown one alternative approach to a handle assembly 702 that includes structure that simplifies a device-user interface so that it can be more intuitive to users. Here, FIG. 12A depicts a cross-sectional view in a home position and FIG. 12B depicts a perspective view of the handle assembly 702 in a home position with one side of the housing being transparent. As stated above, when a handle assembly is in a home position, the treatment device is in the home or sheath configuration (FIG. 2C). FIG. 12C depicts the handle assembly 702 in an open configuration where the treatment device is in an open or hook configuration (FIG. 2E) and FIG. 12D depicts the handle assembly 702 in an active configuration where the treatment device is in an active or cut configuration (FIGS. 2F-G). As shown in the figures, there is no home button in the handle assembly 702, but the handle assembly 702 includes an actuating block 704 that includes a slider 706 with a push surface and an active button 708. The actuating block 704 is configured to translate along with an elongate rod 705 extending longitudinally within the handle assembly 702, and is placed in contact with a return spring 710 configured about the rod 705, the return spring 710 being configured to facilitate returning the actuating block 704 to the home position. The active button 708 further includes an angled slot 712 defining a cam surface that is configured to receive a peg 714. In use, the slider 706 is engaged to translate the actuating block 704 distally so that the treatment device moves from a home or sheath configuration to an open or hook configuration (FIG. 12C). In the home position and open position, the peg 714 is located in a lower proximal position of the angled slot 712. Also, as the slider 706 is advanced forwardly, a release pin 715 operatively associated with the actuating block 704 is translated distally along a track 716 formed in the housing and springs into a vertically oriented slot 717 in the in the track 716 where the release pin 715 is latched until the active button 708 is depressed. Upon depressing the active button 708 (FIG. 12D), structure of the active button 708 that engages the release pin 715 removes the release pin 715 from the slot 717 portion of the track 716 and the treatment device assumes an active or cut configuration. Along with this action, the peg 714 is translated along the angled slot 712 to an elevated and forward position in the slot 712 thus guiding the movement of the active button 708. Once the active button 708 is released, the return spring 710 pushes the actuating block 704 proximally before the release pin 715 can relatch within the slot 717 in the track 716, thereby allowing the actuating block 704 (and treatment device) to return to home (FIGS. 12A-B).

In a related approach (FIGS. 12E-H), the handle assembly 720 can be further provided with a deflectable latching arm 722 that engages complementary latching structure 723 configured within the handle assembly 720 when the actuating block 726 of the handle assembly 720 is in the home position (FIGS. 12E-F) and when the actuating block 726 is advanced along with the elongate rod 705 to an open position (FIG. 12G) by applying a distally directed force on a slider 728. Depressing an active button 730 results in the system assuming an active configuration (FIG. 12H), and the latch arm 722 is deflected out from engagement with the complementary latching structure 723. Contemporaneously, the peg 714 is translated along the cam surface of the angled slot 712 away from its proximal position. This drives the pushrod block (714 and 724) forward to lift the latch off the complementary latching features 723 in the handle halves. The return spring 710 moves the whole actuating block 726 backwards before it can relatch and returns the actuating block 726 to home. (FIGS. 12E-F).

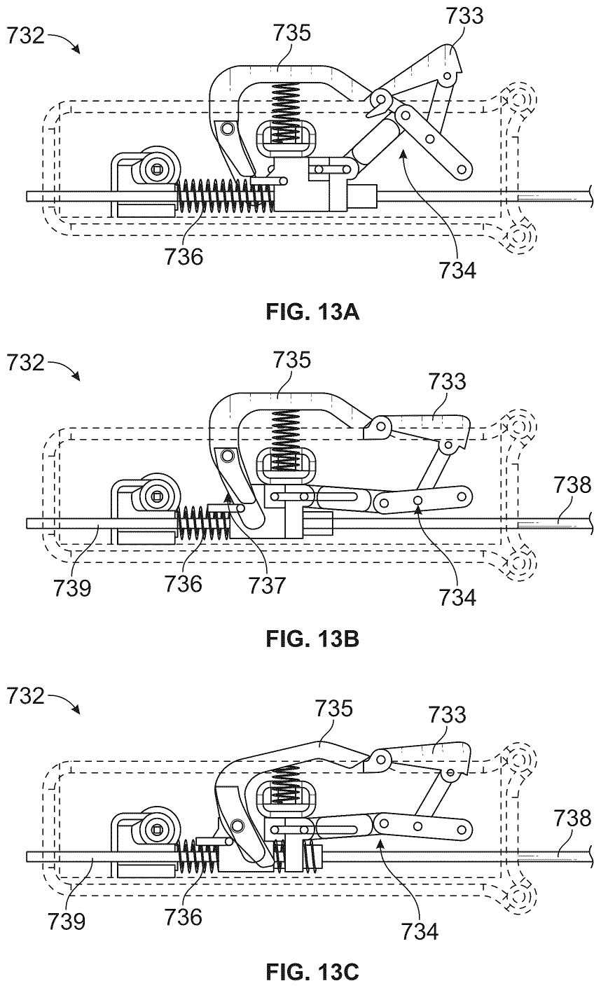

In another two-button approach (FIGS. 13A-C), the handle assembly 732 includes a first button 733 which moves a linkage 734 to deploy a hook (see also FIG. 2E) and a second button 735 to activate a blade (FIGS. 2F-G). When the handle assembly 732 is configured to present a hook at the distal end portion of the assembly, the linkage 734 is moved into an over-center position so that it remains in position while resisting forces provided by a return spring 736 (FIG. 13B). Pressing the second button 734 engages a cam 737 which functions to deploy the blade, while simultaneously moving the linkage 734 into an unstable position (FIG. 13C) so that when the second button 735 is released, the handle assembly returns to a home position (FIG. 13A). Additionally, the handle assembly 732 is configured to move an outer tube 738 relative to a pushrod 739 to present hook and cut configurations. In order to sheath the treatment device configured at the distal end of the assembly when the hook is deployed, the outer tube 738 is moved distally relative to the handle. This has the effect of moving the hook away from any tensioned tissue. By actuating in this manner, the device becomes more immune from tension on the treatment device.

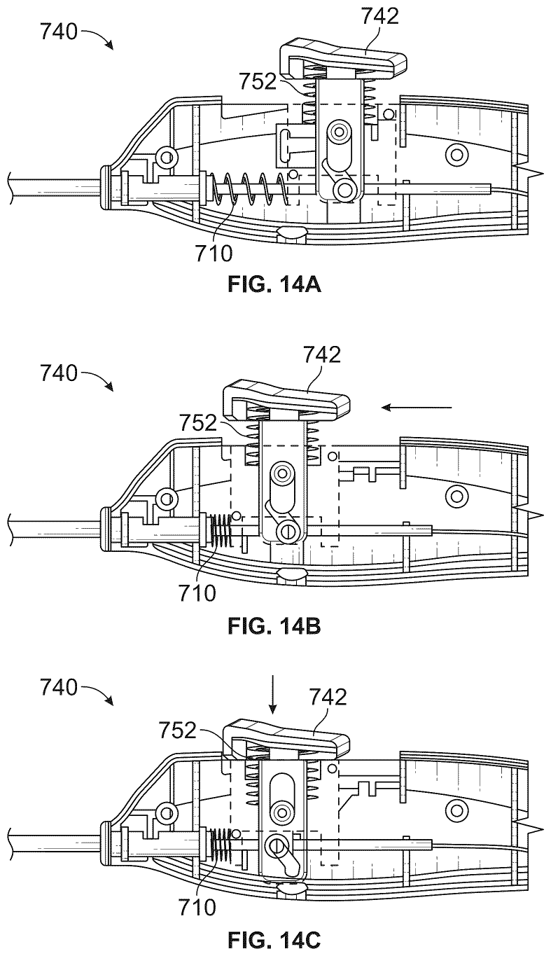

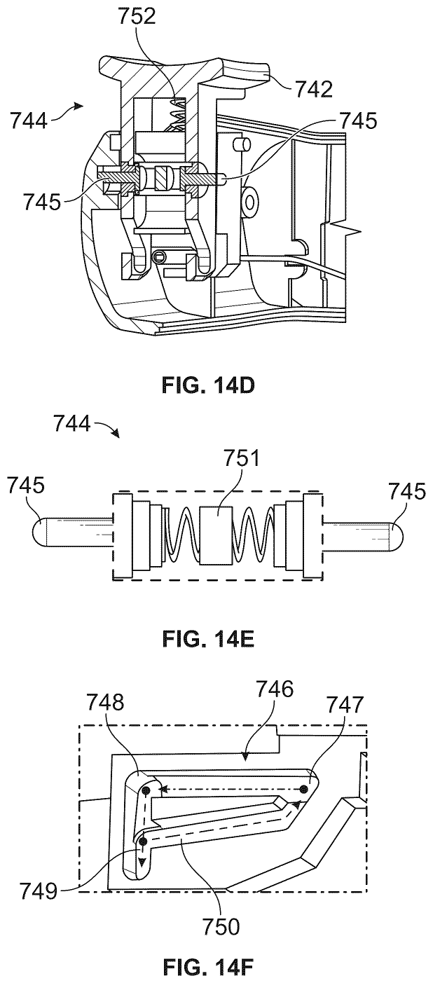

In another approach (FIGS. 14A-E), the handle assembly 740 includes a spring biased actuation button 742 equipped with a horizontally arranged spring pin assembly 744 with opposing follower ends 745 that are each configured to be translated along a generally P-shaped track 746 defining cam surfaces that is formed within opposing sides of the handle assembly 740. The spring pin assembly 744 and P-shaped tracks 746 cooperate to control the configuration of a treatment device into and out of home or sheath (FIG. 2A), open or hook (FIG. 2E), and active or cut configurations (FIG. 2F-G). Notably, each stage of configuration of the treatment device is associated with different adjacent depths of the tracks 746 so that the pins 744 are locked within a portion of the track 746 that is associated with a particular treatment device state. It is to be noted that the relative differences between the various portions of the tracks can be created by ramps and drop-offs so that the differences in the depths are created at junctures between track sections, and the pin follower ends 745 are translated in one direction along smooth ramped surfaces. Accordingly, in a home configuration (FIG. 14A), the pin follower ends 745 reside in a first section 747 of the track 746. When the actuating button 742 is slid forward or distally as far as it can be placed, the follower ends 745 drop into a second portion 748 of the track 746. At this position, the spring 751 of the pin spring assembly 744 maintains the follower ends 745 within an adjacent relatively deeper portion of the track 746 and the treatment system assumes an open or hook configuration. Next, the actuating button 742 can be depressed to move the pin follower ends 745 to a third portion of the track 749 which has a depth that is again different from the adjacent second portion of the track 748, the spring assembly 744 again maintaining the pin follower ends 745 within the third portion of the track 749. Accordingly, when the actuating button 742 is depressed so that the pin followers 745 move to track portion 749, the pushrod block (714) is driven forward and advances the pushrod 705. The track 746 and pins 745 are thus provided to ensure that the slider assembly is in the right position for each of the states. At this point, the treatment device is placed in the active or cut configuration. Upon complete compression and then release of the actuating button 742, the spring 752 of the actuating button 742 causes the pin follower ends 745 to move to a fourth 750 section of the track 745 along which the follower ends 745 are caused to move proximally to the first section 747 of the track 746 and to place the treatment system back into a home or sheath configuration.

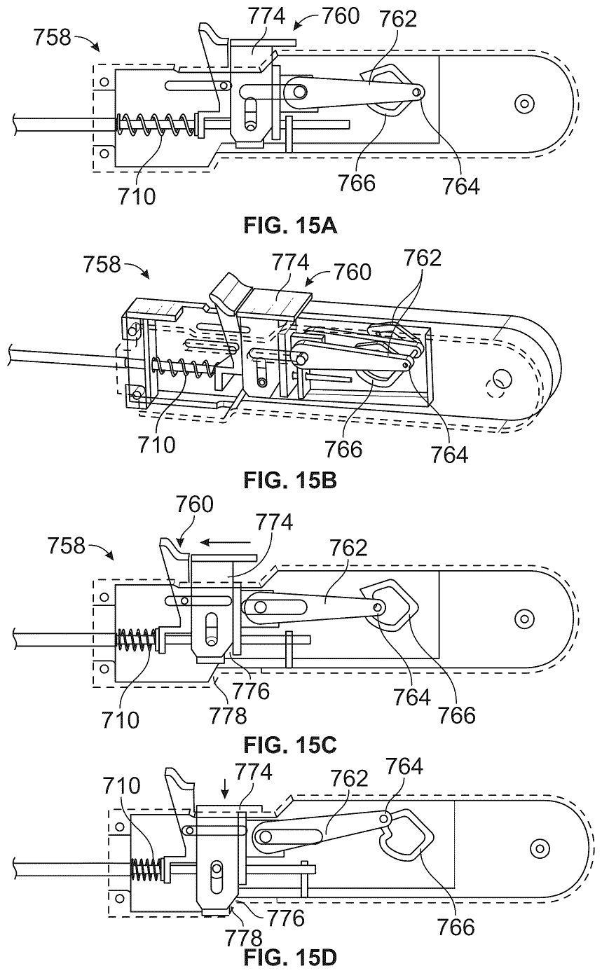

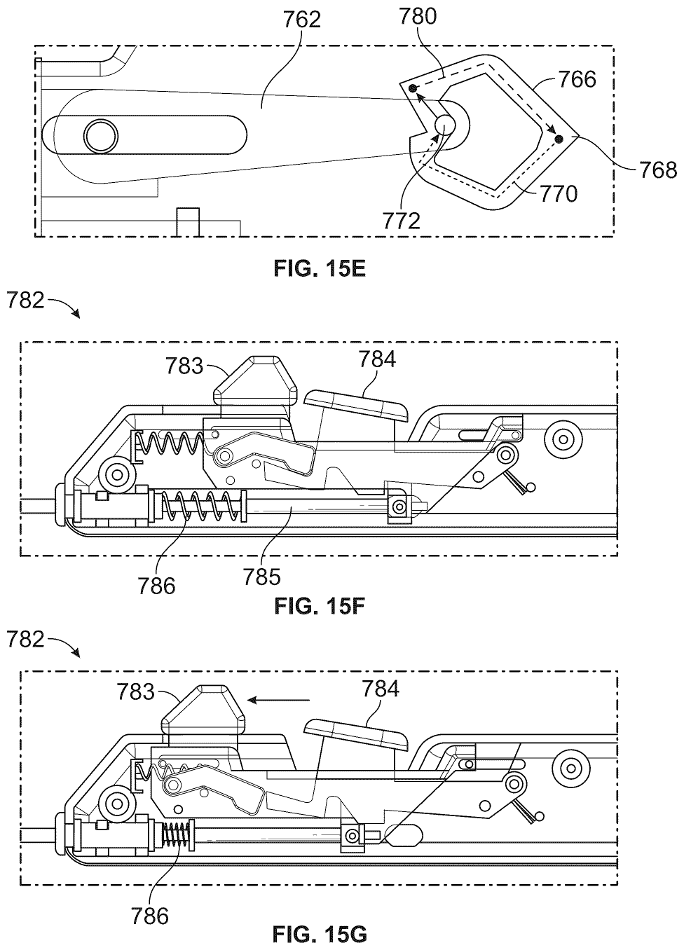

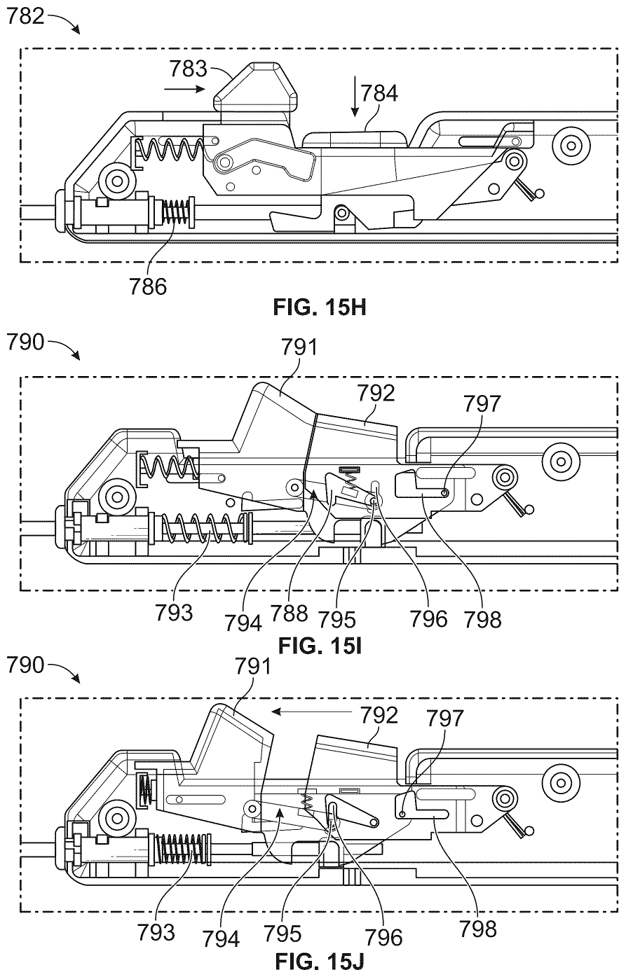

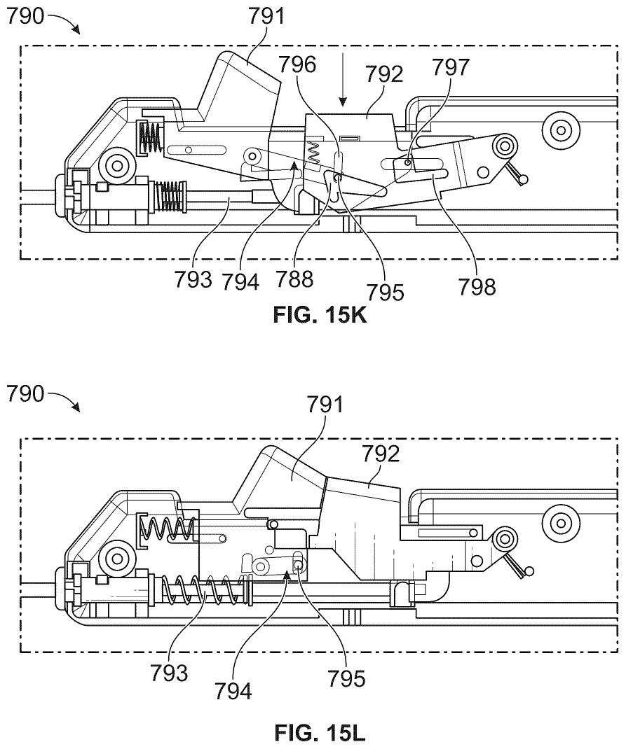

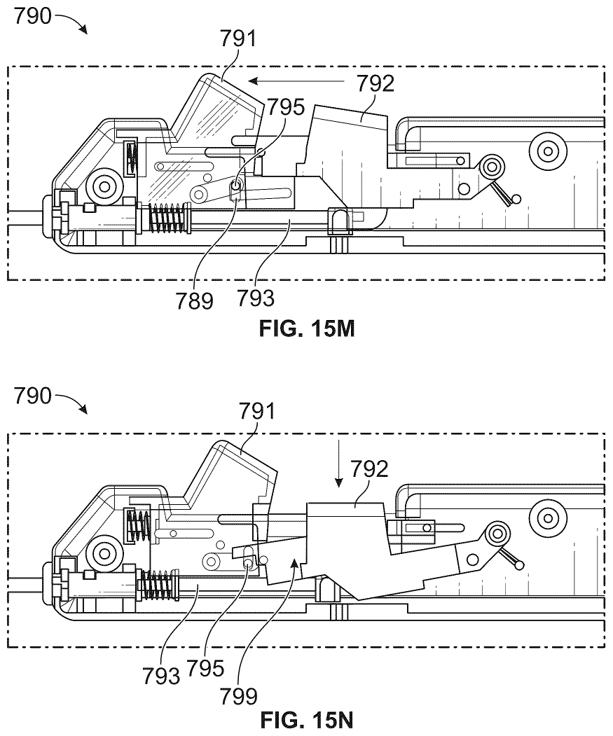

In a related track guided approach to a handle assembly 758 (FIGS. 15A-E), a proximal portion of an actuatable block 760 is equipped with a pair of rotating follower arms 762 that move in concert and each include a terminal end that defines a pin follower 764 that projects generally perpendicular to the follower arm 762. The pin followers 764 are each sized and shaped to be placed within and translate along an irregularly shaped and closed-loop track 766. Friction between the follower arms 762 and their pivot points provides desired control of handle assembly 758 as it cycles between home, open and active states. In a home or sheath position (FIGS. 15A-B), the pin followers 764 are in a most proximal corner 768 of the tracks 766 (See FIG. 15E). Sliding the actuatable block 760 forwardly causes the pin followers 764 to move along a first section 770 of the tracks 766 to a bottom of a v-shaped portion of a second section 772 of the tracks 766 (FIG. 15E). It is here that the treatment device is placed and held in an open or hook configuration (FIG. 2E) and the handle assembly 758 is in an open or hook state (FIG. 15C). Depressing an active button 774, results in the treatment device assuming an active or cut configuration (FIGS. 2F-G) and the handle assembly 758 being placed in an active or cut state (FIG. 15D). A bottom portion of the actuatable block 760 is provided with an angled surface 776 that engages a complementary ramp structure 778 formed within the housing assembly. These structures are arranged to cooperatively ensure that the actuatable block 760 is fully advanced into a cut position. When so placed, the pin follower 764 is removed from the second section 772 of the track 766 (FIG. 15E) so that the actuatable block 760 and the treatment device can return to the home positions (FIGS. 2E-F, 15A-B) through the biasing action of the spring 710. Here, to reach home, the pin followers 764 are each translated along a third section 780 of the tracks 766 (FIG. 15E).

In another approach (FIGS. 15F-H), the handle assembly 782 includes a slider 783 to deploy a hook (see FIG. 2E), and a button 784 to activate a cutting blade (FIGS. 2F-G). Once the button 784 is released, the handle assembly 782 returns to its home position (FIG. 15F). Here, the button 784 does not move forward with the slider 783, but stays in its original position making it easier to reach without the user having to adjust their hand position. The pushrod 785 is not permanently connected to either the slider 783 or the button 784 but is handed off between the two structural components. Once the button 784 is released, the pushrod 785 is freed from both the slider 783 and the button 784, thereby allowing the full potential energy of the spring 786 to work against any residual tip tension that may be present without being hampered by friction imposed by internal handle components. In this way, performance against tissue tension at the distal tip of the assembly is improved.

With reference to FIGS. 15I-N, there are shown additional handle assemblies that are configured to reduce the number of steps or actions required by a user to operate a treatment device. As with previously described approaches, by doing so, potential for user fatigue is diminished and an amount of time required for a user to learn and internalize how to use the treatment device can be reduced. Handle assemblies can function by having three states, namely, home, open and active, where a user must take an action in order to place the device into a next state such as by pushing a button or moving a slider. In certain approaches to handles, the user must take an action to return the handle assembly to a home position. However, in the presently described embodiments, the user action associated with returning the handle assembly to the home state can be eliminated. In a specific approach, the return of the handle assembly to the home position can be made despite tension provided by tissue against the treatment device. This may be necessary when a user has completed a cut, and a blade of the treatment device comes to rest against nearby tissue structures with some amount of tension that can interfere with the mechanism returning to the home position. Eliminating the manual action to return to home also reduces the number of buttons or sliders presented to the user and as stated, facilitates more quickly learning how to use the treatment device. Further, the disclosed handle assemblies prohibit actuating mechanisms to return to the home position until a tissue cutting actuating mechanism is released. This can prevent confusion as to which state the device currently assumes.

As shown in FIGS. 15I-K, a handle assembly 790 includes a spring biased slider 791 and a cut button 792 for manipulating a spring biased pushrod 793 operatively associated with a treatment device (not shown). The pushrod 793 is not permanently connected to either the slider 791 or the cut button 792, but is handed off between the two structures. Once the cut button 792 is released, the pushrod 793 is freed from the slider 791 and cut button 792, thereby allowing a full potential energy of its spring to work against any device residual tip tension without being hampered by friction imposed by the mechanical action of handle components. In this way, performance against tissue tension at a distal tip of the treatment device can be optimized. Here, action of the pushrod 793 is controlled by a spring-loaded latch 794 that includes a projection 795 that rides in a first track 796 formed in the slider 791 and a second triangular shaped track 788 formed in the cut button 792, and a second projection 797 that rides in a third track 798 formed in the cut button 792.

In a home state (FIG. 15I) where the treatment device is in a stowed position, the slider 791 is in a proximal position and the cut button 792 is undepressed. As the slider 791 is pushed forward, the first projection 795 travels within the first track 796 and locks the device in an open state (FIG. 15J). At this point, the treatment device is deployed to present a hook configuration but a blade or cutter of the treatment device is covered or undeployed. Next, when the cut button 792 is depressed (FIG. 15K), the first projection 795 unlatches from the first track 796 but the treatment device is held in the open state by the second projection 797 configured in the third track 798 formed in the cut button 792. Now, the treatment device is in a cutting configuration where the blade or cutter of the treatment device is exposed and can be used to treat target tissue. Upon releasing the cut button 792, the second projection 797 unlatches, allowing the slider 791 to return to its proximal position and the cut button 792 to return to its undepressed configuration. The treatment device thus automatically returns to the stowed configuration and the handle assembly 790 is in the home state (FIG. 15I).

In an alternative and related approach (FIGS. 15L-N), the pushrod 793 is controlled by a spring-loaded latch 794 including a projection 795 that rides in a track 789 (FIG. 15M) formed in the handle 790 itself. Here, as the slider 791 is pushed forward (FIG. 15M), the projection 795 travels forward in the track 789 and locks to hold the treatment device (not shown) in a deployed and open or hook position. When the cut button 792 is pressed (FIG. 15N), the projection 795 unlatches from the track 789 but the treatment device is held in a deployed position by a nub 799 formed on the cut button 792 that interferes with the slider 791. The treatment device is thus simultaneously converted to present cutting structure to tissue. Once the cut button 792 is released, the nub 799 disengages from the slider 791 and the treatment device automatically returns to a stowed configuration and the handle returns to the home state (FIG. 15L).

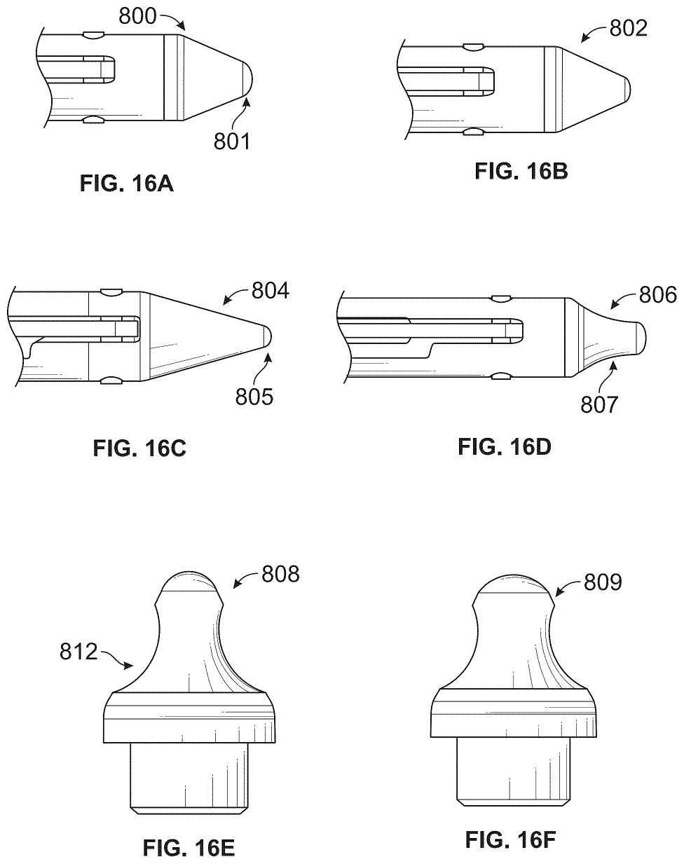

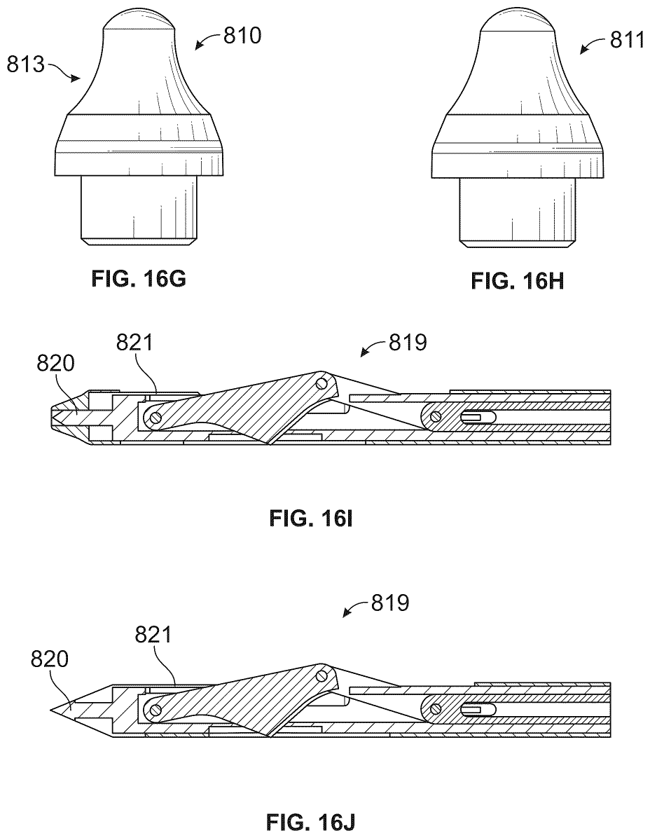

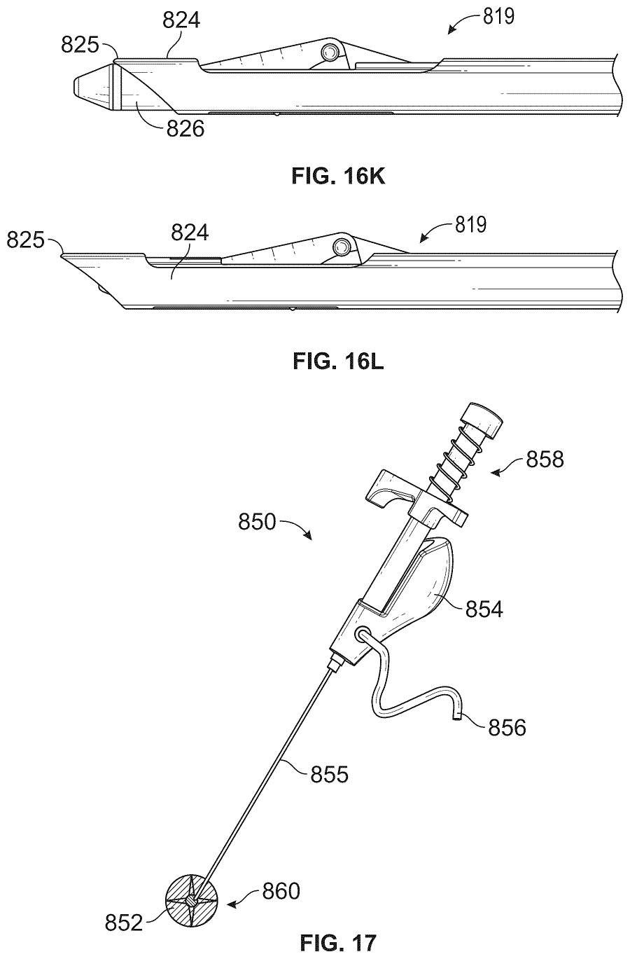

Turning to FIGS. 16A-G, a terminal end distal portion of the treatment device can assume various different profiles designed to facilitate the advancement of a cellulite treatment system to an interventional site in a subcutaneous space. The drive force necessary for tunneling through the subcutaneous lipid layer is reduced, while also maintaining high skin puncture forces. A balance between tip sharpness required to navigate to the interventional target site and avoiding unwanted piercing of tissue is sought. That is, one balance sought is so that a tip is provided that advances easily but does not pose a risk of crossing the skin during use as is associated with a particularly sharp tip. Thus, lowering drive forces is desired and the terminal end of a treatment device can aid in finding the correct balance.

In various approaches, the tip can be a singular machined part that is designed to fit into the outer tube of the treatment device, where it can be laser welded into place. The tip is designed to push through subcutaneous lipid layers with minimal force, but also be shaped such that it requires higher force for skin puncture. Accordingly, the terminal distal portion can have one or more of a generally cone shape 800 (FIG. 16A), a relatively more narrow cone tip shape 802 (FIG. 16B), a relatively longer cone shape 806 (FIG. 16C) or a tip characterized by one or more relief cut-outs 804 (FIG. 16D). Moreover, the tip can embody a thin or thick scalloped profile 808, 809 (FIGS. 16E-F) or a thin or thick teardrop profile 810, 811 (FIGS. 16G-H). The tip profiles of FIGS. 16A-C present an increasingly “sharp” tip profile that corresponds with a decrease in force required to pass through subcutaneous tissue. Tips may be substantially flat (801, FIG. 16A) or sharper (805, FIG. 16C) in order to achieve lower advancing forces. One drawback of a sharper tip is that it becomes easier for the user to unintentionally puncture through a patient's skin. FIGS. 16D-H are intended to reduce the force required to pass through the subcutaneous fat layer without excessively reducing the force required to puncture skin by creating small areas of concavity for septa to “bridge” over as they are stretched around the tip. FIGS. 16D-H all have these areas, which have been highlighted at 807 (FIG. 16D), 812 (FIG. 16E), and 813 (FIG. 16G). This bridging of septa increases the pressure exerted at the very tip of the device, making transit through the septa easier. Skin, not being as elastic as septa tends not to lay against the sides of the tip, so its force model is largely unchanged by the areas of concavity. Additionally, the tips shown in FIGS. 16D-F are not fully revolved shapes, meaning the areas of concavity do not go all the way around the tip but create edges at the boundaries of the cuts. This allows for the user to use a twisting motion to potentially cause septa to part, allowing for easier transit through tissue.

With reference to FIGS. 16I-L, there are shown further approaches to tips for a treatment device. It has been recognized that tunneling through tissue in the superficial subcutaneous space to a target treatment site with a blunt tip can require some users to exert force, especially when navigating through septa. While a pointed tip is effective, there is a risk of unintended piercing of the skin or other tissues with the pointed tip. Thus, deployable and retractable piercing structure can be incorporated into a treatment device to avoid this risk. By doing so, forces required to tunnel through tissues can be selectively reduced to thereby provide a better user experience and improved patient comfort and recovery. In one approach (FIGS. 16I-J), a treatment device 819 is provided with a piercing rod 820 that in a stored configuration is covered by a longitudinally extendable and slidable sheath 821 that extends proximally to a handle assembly (not shown). In an alternative approach, the piercing rod 820 can be replaced with a blade-like structure (not shown). The sheath 821 can be actuated by a cam, a slider or a dial manipulated by cooperating structure incorporated into the handle assembly to thereby project and expose the piercing rod (FIG. 16 J). It can also be spring loaded such that when a high enough force is encountered, the sheath is pushed back uncovering the tip. In an ideal embodiment, this would be momentary until the loading anatomic feature is passed and the load is released. In a similar embodiment, this could involve a lock out mechanism configured on the handle so that in the event the treatment device is in thin tissues or the user suspects there is a chance of encountering the skin, the regular blunt tip would remain present under any load condition. Moreover, the sheath 821 itself can function as a longitudinal stiffener for the treatment device and the assembly presents an advantageous and relatively small profile. In an alternative approach (FIGS. 16K-L), piercing structure is embodied in an external sheath 824 that includes a pointed or sharped tip or edges 825. In a stowed configuration (FIG. 16K), the tip 825 of the of the sheath 824 is protected from tissue by a shaft 826 of the treatment device 819. Advancing the sheath 824, as shown in FIG. 16L, such as by manipulating cooperating structure associated with a treatment device handle assembly, results in exposing the tip 825 to tissue for tunneling purposes.

Turning to FIG. 17, there is shown a light needle assembly 850 configured to facilitate improving anesthesia delivery to an interventional site. It is to be recognized, however, that the light needle can be employed in various other applications for other purposes where facilitating navigation within tissue is desired. In one particular aspect, the light needle assembly 850 can be used in conjunction with a cellulite treatment procedure. The light needle assembly 850 embodies a battery powered light source 852 configured to provide enhanced visualization during an interventional procedure, via transillumination, of the treatment site during anesthesia injection or other procedure. A compartment 854 is provided to house a battery and optics and a tube 856 extending from the compartment 854 is configured to be placed into communication with a reservoir (not shown) containing anesthesia fluid. An anesthetic needle 855 extends longitudinally from the handle assembly 854. Additionally, the handle assembly 854 further includes a spring biased syringe 858 that is provided to reload the syringe once released. In this embodiment, but not required, a dual valve assembly can be configured within compartment 854 thus allowing for the syringe contents to be pushed out of the needle, making the syringe available for reloading with fluid at the end of the tube 856. The light transmitted from the terminal end 860 provides information concerning depth, orientation to bevel and distance from entry of the light needle assembly within and between tissue layers. By so doing, accuracy and injection precision is enhanced as the user is provided with feedback that is used to adjust angles and approaches of the light needle assembly 850 through tissue. In one specific aspect, a tissue or body model can be provided so that the assembly 850 can be used for training purposes. In the case of applying an anesthetic, use of the light needle assembly 850 ensures the treatment field is well anesthetized with minimal amount of additional anesthesia and time. In various approaches, a needle assembly system can include one or more of gauges or flow components such as valves integrated with a pump cooperating with the needle assembly to both control operation and ensure effectiveness of the system. Thus, pain associated with procedures conducted in conjunction with anesthesia can be most effectively controlled using the light needle assembly 850.

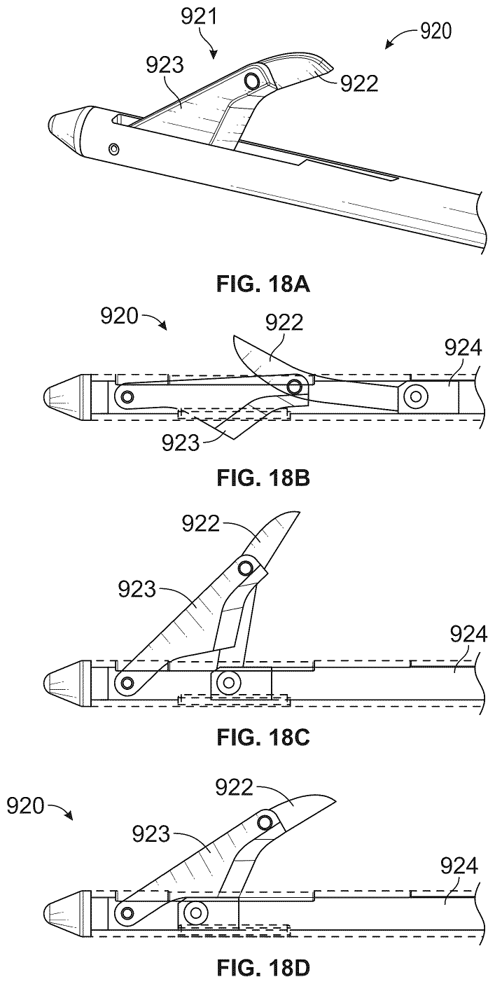

Turning now to FIGS. 18A-D and 19A-D, there are shown additional approaches to treatment devices that include structure that increases an amount of tissue captured by the device, without appreciably increasing the force required by a user to cut captured tissue. By increasing the width of the treatment device, with each pass of the device at a treatment site, time to perform a procedure can be reduced by decreasing the number of passes that must be made by the device. The extension member facilitates capturing tissue and causing the captured tissue to slide toward and along a sharpened blade. As captured tissue enters hook structure, tissue that is already captured slides toward a nook of the hook in order to make room for all captured tissue. This sliding action along the blade enables the blade to better cut captured tissue, thereby reducing the force required to effect a cut.

In a first approach (FIGS. 18A-D), a treatment device 920 includes a hook and cutting subassembly 921 includes a blunt blocker link 922 pivotably attached to a blade link 923. In a sheathed configuration (FIG. 18B), the device can be advanced to a treatment site as described above. A longitudinally extending pusher member 924 operatively associated with the hook and cutting subassembly 921 is configured to repeatably place the subassembly in sheathed, hook and cut configurations as needed and as directed by a user. The blocker link 922 extends beyond a length presented by the blade link 923 to thereby provide additional structure for capturing tissue when the hook and cutting subassembly 921 is placed in the hook configuration. (FIGS. 18A and C). The pusher member 924 is further manipulated such as by advancing the member distally to convert the hook and cutting subassembly 921 into a cut configuration (FIG. 18D). As shown, in such a configuration, a length of the blocker link 922 remains extending beyond the blade link 923 when the hook and cutting subassembly 921 is in the cut configuration. In the cut configuration, the device 920 is manipulated as desired to cut captured tissue while also taking advantage of the sliding of tissue along the blade link 923 to accomplish cutting. The device is then again placed in sheathed and hook configurations to treat additional targeted sites.

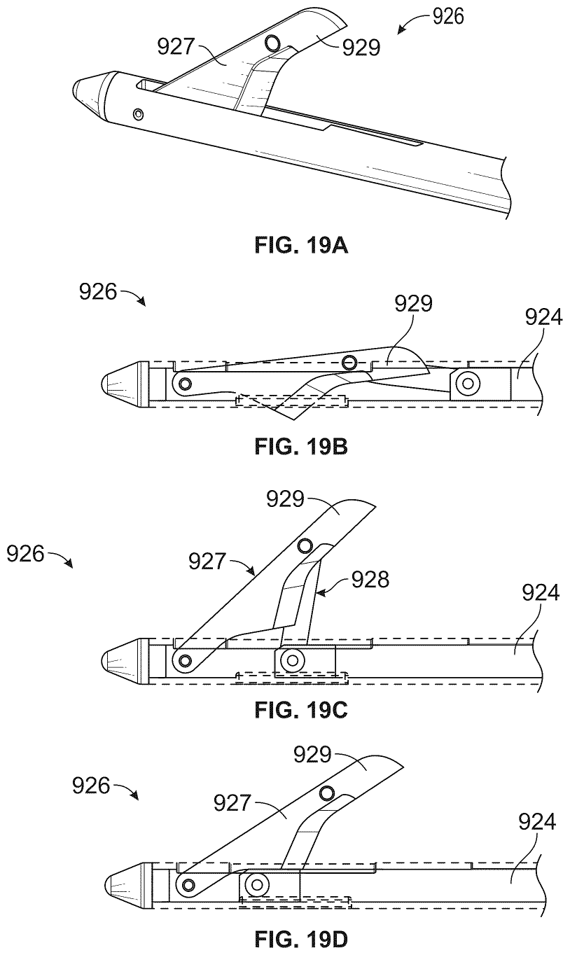

In a related approach (FIGS. 19A-D) to structure that increases an amount of tissue captured by the device without appreciably increasing the force required by a user to cut captured tissue, the treatment device 926 includes a blade link 927 pivotably attached to a blocker link 928. Here, the blade link 927 includes an extension 929 that is not bladed, but presents a blunt surface for capturing tissue. Although the extension is shown in-line with the blocker or blade, it is to be recognized that in this and other embodiments, that the extension structure can be angled to encourage captured or held tissue to press or slide across the blade. In a sheathed configuration (FIG. 19B), the treatment device 926 can be advanced to a targeted treatment site as described above. Here also, a longitudinally extending pusher member 924 operatively associated with the hook and cutting subassembly 930 defined by the blade and blocker links 927, 928 is configured to repeatably place the subassembly in sheathed, hook and cut configurations as needed and as directed by a user. Advancing the pusher member 924 to a first distal position places the hook and cutting assembly 930 into a hook configuration (FIG. 19C). In the hook configuration, the blade extension 929 and blocker link 928 are presented to capture tissue. Notably, the blade extension 929 extends beyond the length of the blocker link 928, the structures cooperating to capture an increased amount of tissue. Further advancement of the pusher member 924 places the hook and cutting assembly 930 into a cut configuration (FIG. 19D), while the blade extension remains deployed to maintain the capture of tissue. In the cut configuration, the device 926 can be employed to cut tissue as directed by the user and the sliding action of tissue along the blade facilitates the cutting of tissue. Thereafter, the device 926 can be placed in sheathed and hook configurations as desired to move the device 926 to further treatment areas or to remove the device from the body of the patient.

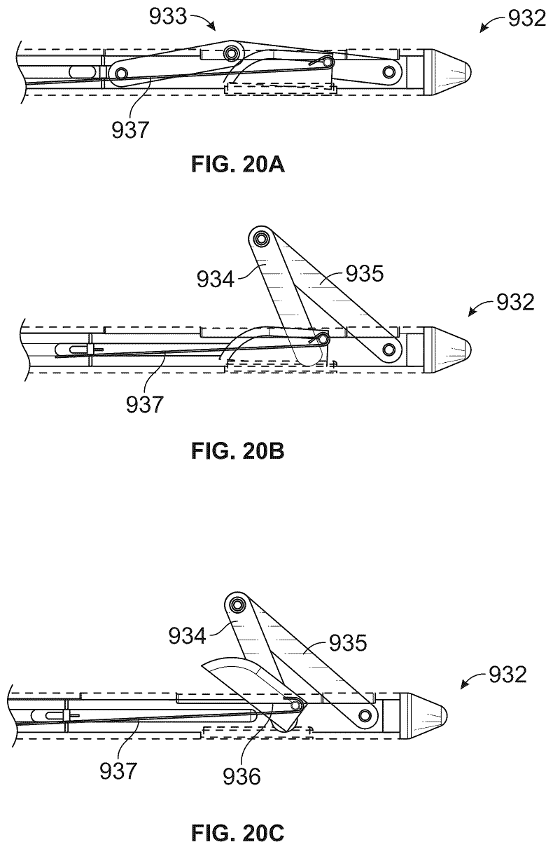

With reference to FIGS. 20A-C, there is shown yet another approach to a treatment device 932 that employs slicing action to cut tissue. In one aspect, the device 932 separates cutting mechanism structure from hooking, tension and verification mechanism structure. The device is also characterized by requiring lower forces to cut tissue such that handle tension is for depression verification only. By taking this approach, the cutting blade can be a more simple and less costly structure.

As shown, the treatment device 932 includes a folding linkage system 933 where both links 934, 935 present blunt or dull surfaces used to capture and tension tissue. Manipulation of the linkage system 933 can be controlled by a longitudinally extending pusher member operated by a user. A separate blade element 936 is provided and actuated by a pull wire 937 also operated by a user. The blade element 936 is deployed after tissue has been captured and tensioned. The pull wire 937 is then manipulated to move the blade 936 through a scissor-like cutting action against the captured and tensioned tissue.

In use, the treatment device 932 is placed in a sheathed configuration (FIG. 20A) to be placed within and between tissue layers of a patient and also to be advanced to a treatment site. Next, the device 932 is placed into an open or hooked configuration to capture and also tension tissue (FIG. 20B). Thereafter, the blade element 936 is actuated and deployed by manipulating the pull wire. The blade element 936 cuts through captured tissue with a slicing and scissor action as the blade element 936 moves across the folding linkage system 933. The device 932 can then be placed in one or more of the sheathed, open or cutting configurations and moved as deemed necessary to complete a treatment procedure. In alternative approaches, the blade can be actuated as a secondary linkage set and/or be operable by a pusher member instead of a pull wire. Also, the blade can be spring-loaded to actuate once a tension wire is released and then pulled back to a home position. Consequently, the blade can be tensioned to cut, and spring-loaded back to the home position.

In addition to treating cellulite, the disclosed devices can be employed in various approaches to treating other areas and conditions. In one or more aspects, the disclosed devices can be used in scar release, acne subcision, facial fold and/or facial lift procedures, or in any area where tissue disruption is needed in the subcutaneous space between tissue layers of the body. Transillumination can be used or specific devices or systems can lack transillumination structures or assemblies. Moreover, certain approaches can lack blocker structure where more simplified tools may be required or desired. This allows for approaches that can tension targeted tissue with a blade edge to perform assessments prior to increasing a load to the level of cutting with the blade edge. Here, target tissue and blade sharpness have an influence on device design.

The assembly can be introduced close to a treatment site or it can allow for moving a tissue entry point to a more aesthetically appropriate or desirable location. With an exposed cutting blade, septa or scar tissue can be engaged and depending on the selected sharpness of the blade, the blade or assembly can otherwise be tensioned to ensure the correct tissue is being treated or directly disrupted. With a hook configuration, the blade can be used to make a verification pass to ensure that all tissue in a target area has been released as desired. In various related embodiments, ports or lights can be added for transillumination or navigation within tissue, and features such as supporting or strengthening materials or structures can be added as there may not be a strict size limitation on entry. In one specific approach, the assembly can be approximately six inches in length and elongate assembly having a diameter of approximately 2.7 mm.

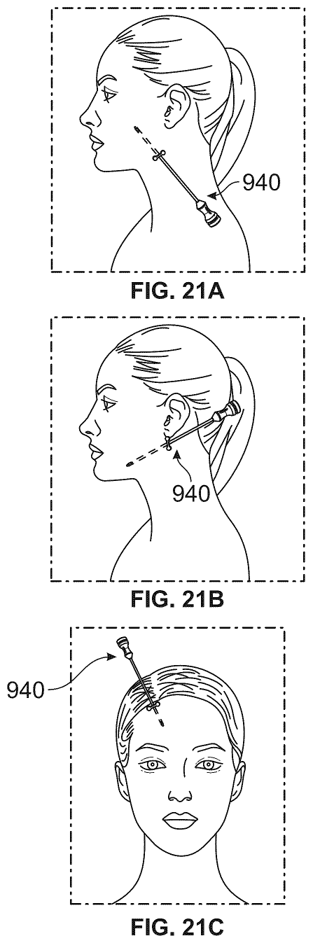

In conventional acne or scar or other procedures, treatment devices are required to be inserted within tissue close to the interventional site, and travel within tissue to the site results in cutting or disrupting non-target tissue. In the present treatment approaches, a treatment device can be placed into tissue at more cosmetically desirable locations where the treatment device includes a blade that is only exposed once it reaches the treatment tissue. In addition, a single entry site can be used to treat multiple target locations through the single entry site since safe and minimally traumatic navigation between entry and the targeted tissue is possible. In treating the face for example, various approaches can be taken (See FIGS. 21A-C). In one approach, a treatment device 940 can be inserted along the jaw line from various angles such as directed generally upwardly from an insertion site at a corner of the jaw (FIG. 21A) or more laterally or downwardly from an insertion site just below the ear (FIG. 21B). For facelift or facial fold procedures specifically or other procedures, the treatment device 940 can be placed between tissue layers through an insertion site along the hairline (FIG. 21C) for minimal disruption to the face.

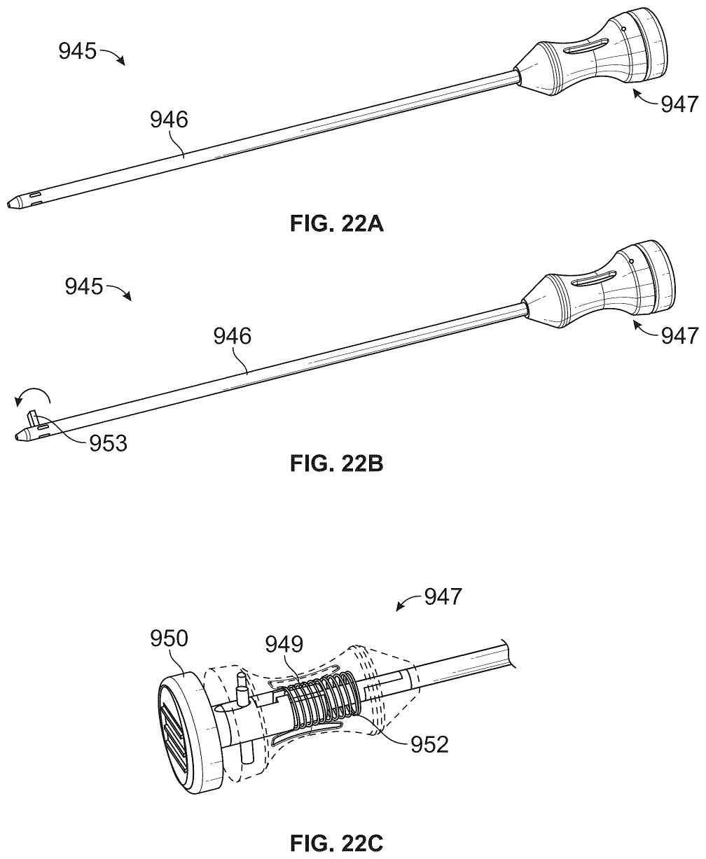

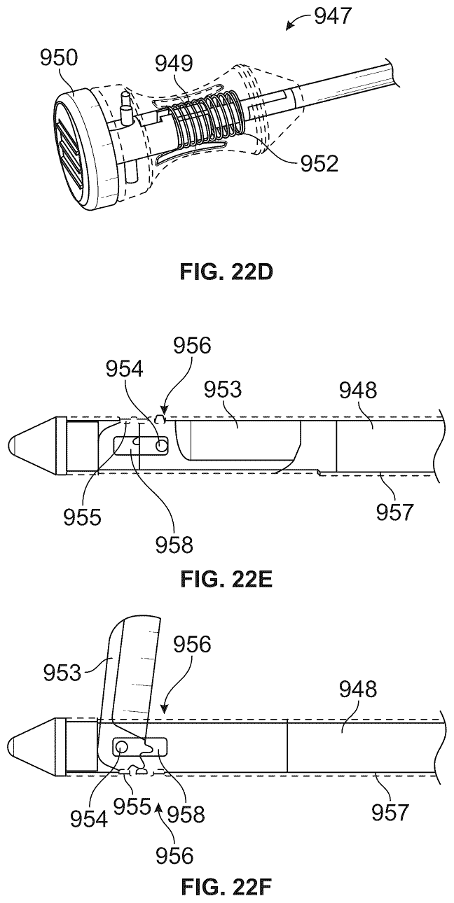

With reference to FIGS. 22A-F, there is shown a simplified treatment assembly 945 configured for treating cellulite or scars in a subcision approach, or in connection with other procedures. The assembly 945 has a length chosen to allow an entry within or between tissue layers some distance away from a target area. The assembly includes an elongate assembly 946 extending from a handle assembly 947 positioned at a proximal end of the elongate assembly 946. Extending within or along the elongate assembly 946 is a pushrod 948. The handle assembly 947 includes a first part 949 and a second part 950 that is biased by a spring 952 the second part 950 configured to be moved longitudinally relative to the first part 949 upon applying a force to the second part 950 that overcomes the spring 952. The spring 952 returns the second part 950 to home upon release of the applied force. The second part 950 is operatively associated with the pushrod 948 so that applying a force to the second part 950 causes the pushrod 948 to translate distally.

Other approaches to handle assemblies for each of the disclosed embodiments can be employed to actuate the blade. For example, the handle assemblies shown in FIGS. 3D-H can be incorporated into the disclosed devices and configured to actuate treatment components. In particular, the treatment device can be configured with a finger ring or other structure that could be manipulated to move a cover distally to create structure designed for blunt, atraumatic dissection or other desired tissue engagement. Such tissue engagement would be similar to dissecting with Metzenbaum scissors where the scissors are advanced closed and then opened within tissue to spread and separate tissue.

As best seen in FIGS. 22B and F, a flip-out blade 953 is operatively and pivotably attached via a pin 954 to a distal end of the pushrod 948. The blade 953 includes teeth 955 configured to be received within corresponding recesses or openings 956 formed in an outer tube 957 of the elongate assembly 946 to create a rack to drive arrangement. The pin 954 translates within a slot 958 as the pushrod 948 is manipulated. This arrangement defines a low profile drive mechanism that allows for blades to have a width near the full width or diameter of the outer tube 957 when stowed. Also, since the blade is stowed along a length of the device, blade length can be chosen without limits and for specific targeted procedures. As the pushrod 948 is advanced, the blade 953 begins to rotate and deploy from a stowed position within the outer tube 957, the engagement of the teeth 955 within the openings 956 and pin 954 with the slot 958 controlling the deployment motion of the blade 953. Deployment can continue until a hard stop is hit such as when the pin 954 engages an edge of the slot 958 or the blade 953 engages the outer tube 956. In one approach, the blade 953 presents a cutting surface configured for performing a drag cut along tissue. The assembly can also be configured to present structure for use in an active cut application, where the blade 953 is manipulated to create a scissor cutting action in combination with structure of the treatment device to which the blade 953 is attached, such as pushrod 948.

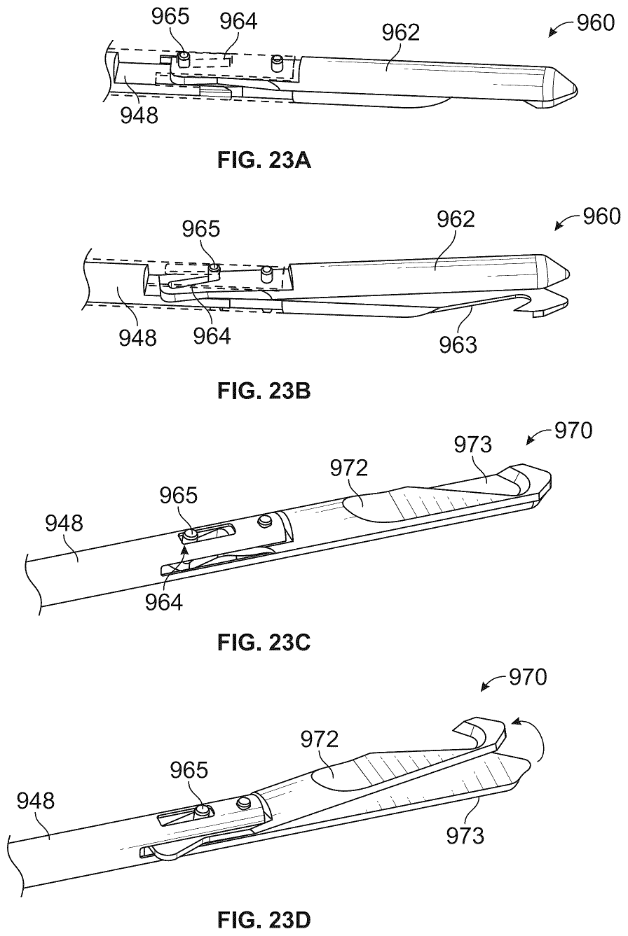

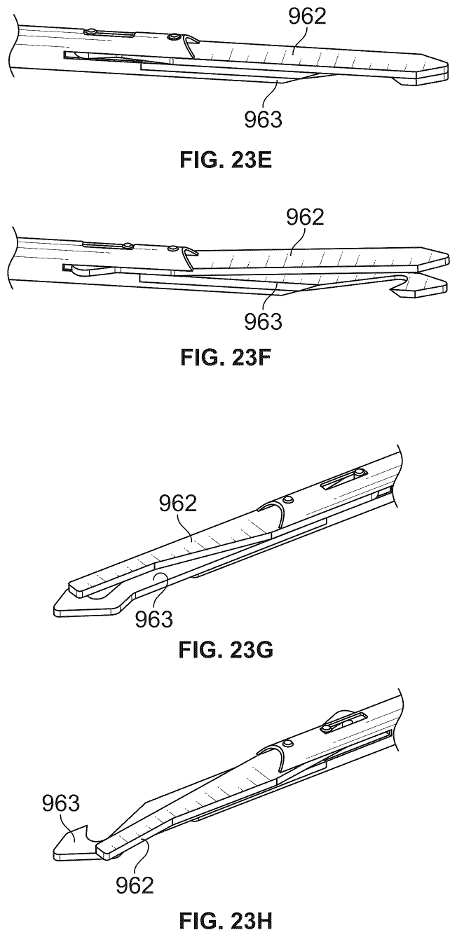

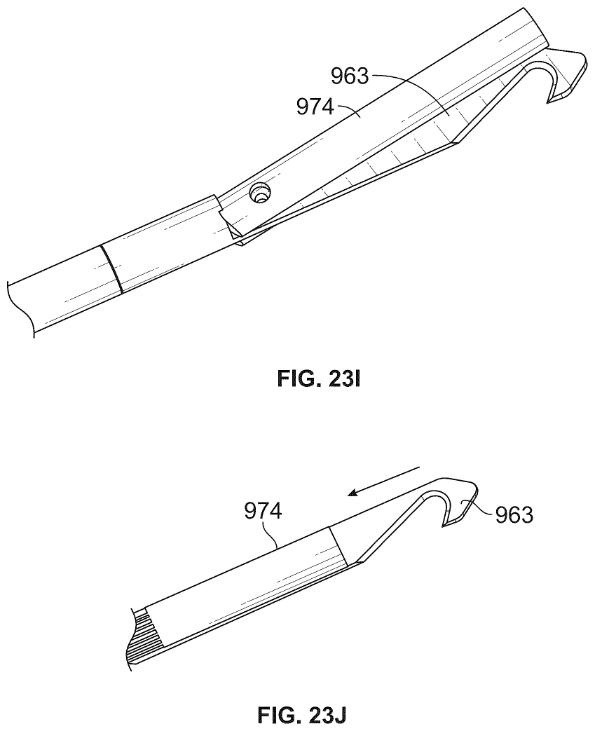

Turning to FIGS. 23A-B, in another embodiment, the treatment assembly 960 includes a rotatable or pivoting cover 962 and a bladed hook 963 provided at a distal end of the elongate assembly, the cover 962 being responsive to the action of the pushrod. The treatment assembly 960 can be placed in a stowed position (FIG. 23A) where the bladed hook 963 is covered by the cover 962, as well as a deployed position (FIG. 23B) where the bladed hook 963 is exposed and available to be used in a treatment procedure. In use, the bladed hook 963 is in the stowed position until the assembly is placed at or near the target area and treatment is desired, thus providing smooth, minimally traumatic advancement within the target tissue plane. In order for the cover 962 to be responsive to the action of the pushrod 948, a cam path 964 is provided in the cover 962, the cam path 964 being configured to receive a pin or cam follower 965 projecting laterally from the pushrod 948. Depressing the second part 950 of the handle assembly 947 results in manipulating or advancing the pushrod 948 (See also FIGS. 22C-D). As this occurs, the cam follower 965 advances along the cam path 964 thus causing the cover 962 to rotate and uncover the bladed hook 963. Releasing the second part 950 allows the spring 952 to return the second part 950 to an undepressed position and the pushrod 948 moves proximally. The cam follower 965 also moves proximally within the cam path 964 causing the cover 962 to cover the bladed hook 963. The bladed hook 963 can be so exposed and covered as needed to accomplish desired cellulite or scar or acne or other treatments. With a modification to the cam path, the cover could also be configured to move in the opposite direction (not exposing the hook). This scissor-like separation of components of the device could be beneficial in blunt (Metzenbaum-like) dissection of tissue.

In a related approach (FIGS. 23C-D), the treatment assembly 970 includes a bladed hook 972 that is configured to deploy by pivoting or rotating away from a cover 973, rather than the cover rotating to expose the bladed hook. This assembly 970 also includes a pushrod 948 that is responsive to actuation of the second part 950 of the handle assembly 947 (See also FIG. 22). Here, the bladed hook 972 is operatively associated with the cam follower 965 that moves along the cam path 964 provided in the bladed hook 972. Advancement of the pushrod 948 causes the cam follower 965 to move within the cam path 964 and to rotate the bladed hook 972 between stowed and deployed configurations.

As shown in FIGS. 23E-F, one or both of the cover 962 and the bladed hook 963 can define flattened profiles. In this way, the treatment device presents a smaller device for treatment. Additionally, as shown in FIGS. 23G-H, the cover 962 can assume various shapes such as a structure that is narrowed at its terminal end. It is to be understood that the bladed hook can also define various profiles suited for a particular purpose.

As shown in FIG. 231, the cover 974 also can be a generally tubular structure with a slot accommodating the bladed hook 963 and that pivots to alternatively cover or expose a bladed hook 963. Alternatively (FIG. 23J), the cover 974 can be a tubular structure that is configured to move longitudinally to alternatively cover or expose a bladed hook 963. In yet a further approach (not shown), the bladed hook structure can include a hinge that is spring-loaded and/or formed from shape memory material such as nitinol. Here, a tubular or other structure is alternatively advanced or withdrawn over the hinge to control the lateral deployment of the bladed hook, and also to stow away the bladed hook in general longitudinal alignment with the treatment device.

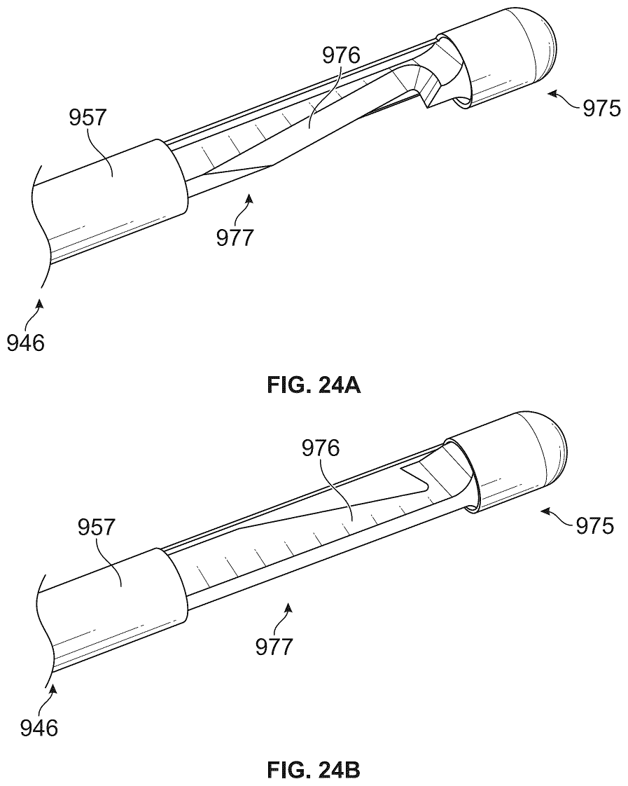

Turning to FIGS. 24A-B, a treatment assembly 975 can embody an axially rotatable bladed hook 976. Here, the handle assembly (not shown) is equipped with structure that accomplishes the rotation of the bladed hook 976. The bladed hook 976 can be configured to rotate within the elongate assembly 946, and a window, cut-out, or opening 977 is configured in the outer tube 957 of the elongate assembly 946. The length of the bladed hook 976 can be commensurate with the opening 977 to thus expose the bladed hook 976 to targeted tissue when it is in a deployed configuration. When stowed (FIG. 24B), the bladed hook 976 is protected from tissue by the outer tube 957. When deployed (FIG. 24A), the bladed hook 976 is exposed to tissue through the opening 977. As with each of the disclosed embodiments, the bladed hook 976 can assume various profiles as dictated by a treatment procedure.

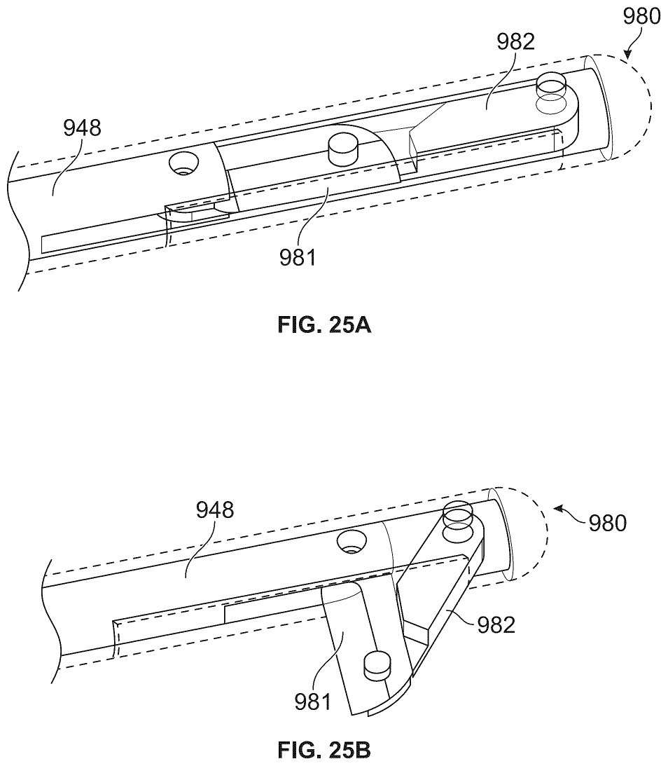

With reference to FIGS. 25A-B, there is shown another approach to a device that can be used in scar release, acne subcision, facial fold and/or facial lift procedures, or in any area where tissue disruption is needed in the subcutaneous space between tissue layers of the body. The treatment device 980 embodies a pushrod 948 operatively connected to a blade 981. The blade 981 is pivotably attached to a blade support link 982 that includes structure configured to support the blade 981 when the blade 981 is in a deployed state. As the pushrod 948 is advanced, the blade 981 pivots from a fully stowed position (FIG. 25A) to a deployed configuration (FIG. 25B). When deployed, the support link 982 provides support to the blade 981 and the blade 981 and support link 982 presents a bladed hook arrangement for engaging targeted tissues.

After completing treatment of one target area employing one or more of the disclosed treatment devices, the procedures described herein can repeated to treat other target areas. Accordingly, the same devices can be employed to access tissue layers below other sites or depressions existing in skin. Notably, in one embodiment, the devices are capable of anesthetic delivery as needed or desired when progressing to additional or new locations. There is thus provided a system configured to treat all target areas on the body through a limited number of small entry sites, including through a single entry site on each side of the patient. It is to be recognized that the system can further include structure permitting the assembly to be steerable to subcutaneous treatment sites. In such an embodiment, the device would be configured to define longitudinally flexible material, and the instrumentation would be steered to the desired position within tissue. Moreover, in certain applications, the device has a stiffness that varies along its length. In another embodiment, the treatment and fat collection devices are embodied in deflectable catheters.

In each of the disclosed approaches and apparatus, should engagement of such septa result in some change in the dimple or depression expressed on the skin, the treatment structures are manipulated to disrupt, cut or slice the tissue. Thus, the treatment structures are opened and tissue is placed between its cutting structure. Next, the cutting structures are advanced against or caused to be closed about the tissue to thereby cut, slice or sever the tissue, thus relieving the tension between tissue layers and eliminating or minimizing the appearance of the unwanted feature on the skin. Actuation is accomplished from a proximal end of the treatment device such as by pulling a wire or advancing and pushing an elongate member associated with the scissor arrangement. Illumination can be provided by a light configured proximal of the treatment or fat collection devices so that transillumination can be employed to track the location of the distal portion of the treatment assembly. Additionally, or alternatively, in each disclosed embodiment, illumination can be via a lightguide from an external light source or via one or more LEDs. Illumination aids the user both with locating the treatment device as well as proper depth placement as transillumination decreases with increasing tool depth. In one aspect, the amount of illumination is set to ensure proper depth of a treatment device or structure, the level of illumination targeted being adjusted for skin type, thickness, presence of fat and pigment.

In employing one or more of the disclosed embodiments in a treatment procedure, there is an expectation that there are instances where it is preferable to not disrupt a hooked tissue, and in such a case it is desirable to release or disengage the hooked tissue. In certain approaches, to release or disengage, the treatment device would be advanced or twisted away from the hooked tissue. It is thus recognized that a challenge exists in that there may be additional tissue in the area which could be unintentionally re-engaged by the treatment device when it is in a hooking configuration, and stowing of the treatment device may be inhibited by adjacent patient anatomy.

Accordingly, various approaches to aesthetic treatment methods and apparatus are presented. The disclosed approaches are configured to provide an effective and focused approach to treating, minimizing and preventing unwanted skin features. The disclosed approaches can also be used to repair and reduce the appearance of unwanted features in a targeted manner. Further, the disclosed proactive treatment modalities are easy and effective to use.

Some of the specific aspects of the present disclosure include one or more of focal treatment of just the tissue responsible for causing unwanted features in the skin; minimizing bruising; accessing all treatment targets from limited, cosmetically acceptable entries; capture and retention of tissue while separating the tissue; intra-operative confirmation of the treated target; needle-diameter sized tools for small openings; and transillumination identification of tool tip location.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A method involving an aesthetic treatment system for treating appearances of a patient's skin associated with a treatment site, comprising:
gaining access to a location between tissue layers connected by septa;
advancing a mechanical septa disrupting element that cooperates with a septa hooking element to a target site;
deploying the septa hooking element to a first position, which shields the septa disrupting element;
engaging selected septa to determine if the selected septa area associated with an appearance of cellulite;
transitioning the septa hooking element to a second position to expose the septa disrupting element;
separating certain of the selected septa that are associated with the appearance of cellulite; and
inserting a fat collector assembly at the target site, the fat collector assembly defining a cannula with a distal portion forming a basket configured to disrupt or collect fat tissue.

2. The method claim 1, further comprising creating a treatment regimen involving using a same insertion site to treat multiple areas.

3. The method claim 1, wherein the septa hooking element operates to tension selected septa.

4. The method claim 1, further comprising employing transillumination to track positioning of the aesthetic treatment system between tissue layers.

5. The method claim 1, further comprising providing the mechanical septa disrupting element with a shaft sized and shaped to be inserted within tissue and to be advanced between the tissue layers to a septa treatment site without assistance from tissue stabilizing structure applied to the patient's skin.

6. The method claim 1, further comprising providing the aesthetic treatment system with tissue cutting, slicing or disrupting structure and manipulating the tissue cutting, slicing or disrupting structure to cut, slice, disrupt, re-orient or rupture septa connecting tissue layers.

7. The method claim 1, wherein one or more of the mechanical septa disrupting element or fat collector assembly define steerable catheters.

8. The method claim 1, further comprising employing a vacuum pump connected to the fat collection assemble to facilitate removing fat tissue from the treatment site.

9. The method claim 1, further comprising providing a plurality of aesthetic treatment devices, combining the devices into a combined single assembly, and conducting an aesthetic treatment procedure with the combined single assembly.

10. The method claim 1, further comprising confirming that the certain of the selected septa that are associated with an appearance of cellulite are separated.

11. The method of claim 1, further comprising, if septa remain intact that are associated with an appearance of cellulite, engaging additional selected septa to determine if the additional selected septa area associated with the appearance of cellulite; and
separating certain of the additional selected septa that are associated with the appearance of cellulite.

12. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure; and
an elongate fat collector assembly configured to disrupt or remove fat tissue from a treatment site.

13. The system claim 12, wherein the fat collector assembly includes a cannula and a basket configured at a distal end portion of the cannula, the basket configured to disrupt and collect fat tissue.

14. The system of claim 12, wherein the fat collector assembly includes a vacuum pump for applying a vacuum to remove fat tissue from a treatment site.

15. The system of claim 13, wherein the basket is collapsible and expandable.

16. The system of claim 12, wherein the fat collector assembly includes a single tube having a series of slots configured in a distal end portion of the tube that define expandable and collapsible flex members.

17. The system of claim 16, further comprising a core shaft that is configured within the single tube and connected to a distal end of the tube to control expansion of the flex members.

18. The system of claim 12, further comprising an outer tube configured to be rotated about an inner tube and as it is rotated, a tip of the fat collector assembly is drawn proximally such that flex members configured at a distal end portion of the fat collector assembly are deflected outwardly to an amount dependent on the relative motion of the inner and outer tubes.

19. The system of claim 12, wherein the mechanical septa disrupting element defines a scythe assembly including a curved blade that both rotates into position and penetrates tissue.

20. The system of claim 12, wherein the mechanical septa disrupting element includes a deployable hook and cutting assembly in combination with a cylindrical reciprocating or rotating sleeve that includes a ground or sharpened distal end.

21. The system of claim 12, wherein the mechanical septa disrupting element includes a hook and cutting subassembly that cooperates with a pair of projectable links, at least one of the projectable links including a cutting edge to thereby provide an actuated cut functionality.

22. The system of claim 12, wherein the mechanical septa disrupting element includes a cutting assembly that accomplish septa severing through a sliding action.

23. The system of claim 22, wherein the cutting assembly includes a hook nook and a cut nook.

24. The system of claim 22, further comprising blocking and cutting structures and a tensioned wire actuation arrangement that is employed to control the blocking and cutting structures.

25. The system of claim 12, further comprising a reciprocating blade assembly in combination with a hook and cutting subassembly.

26. The system of claim 25, wherein the reciprocating blade assembly includes a reciprocating serrated blade.

27. The system of claim 25, wherein the reciprocating blade assembly includes a reciprocating straight edge.

28. The system of claim 12, further comprising include two sets of deployable hook linkages connected to a shaft and projecting at opposite sides of the shaft.

29. The system of claim 12, further comprising a touch-up treatment assembly that is used to treat areas subsequent to a primary treatment session.

30. The system of claim 29, wherein the handle includes one or more of a syringe handle arrangement, a pistol grip actuation assembly, a forceps actuation assembly or a control syringe handle assembly that includes a hoop for grasping by an operator.

31. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure;
wherein the mechanical septa disrupting element defines a scythe assembly including a curved blade that both rotates into position and penetrates tissue.

32. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure;
wherein the mechanical septa disrupting element includes a deployable hook and cutting assembly in combination with a reciprocating or rotating structure that includes a ground or sharpened distal end.

33. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure;
wherein the mechanical septa disrupting element includes a hook and cutting subassembly that cooperates with a pair of projectable links, at least one of the projectable links including a cutting edge to thereby provide an actuated cut functionality.

34. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure;
wherein the mechanical septa disrupting element includes a cutting assembly that accomplishes septa severing through a sliding action.

35. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure; and
a reciprocating blade assembly in combination with a hook and cutting subassembly.

36. An aesthetic treatment system for treating appearances of a patient's skin associated with a treatment site, comprising:
an aesthetic treatment assembly including an elongate member extending from a handle and configured to be insertable through the skin and a mechanical tissue disrupting element attached to a distal end of the elongate member that is capable of expanding at least one region from a smaller state to a wider state and engaging one or more regions of tissue subcutaneously, wherein the mechanical tissue disrupting element is configurable to both define tissue hooking structure as well as tissue severing or disrupting structure; and
two sets of deployable hook linkages connected to a shaft and projecting at opposite sides of the shaft.

37. An aesthetic treatment system for treating appearances of a patient's skin associated with a treatment site, comprising:
an elongate fat collector assembly configured to disrupt or remove fat tissue from a treatment site, wherein the fat collector assembly includes a cannula and a basket configured at a distal end portion of the cannula, the basket configured to disrupt and collect fat tissue and wherein the basket is collapsible and expandable.

38. The aesthetic treatment system of claim 37, comprising a single tube having a series of slots configured in a distal end portion of the tube that define expandable and collapsible flex members and a core shaft that is configured within the single tube and connected to a distal end of the tube to control expansion of the flex members.

39. The aesthetic treatment system of claim 37, comprising an outer tube configured to be rotated about an inner tube and as it is rotated, a tip of the fat collector assembly is drawn proximally such that flex members configured at a distal end portion of the fat collector assembly are deflected outwardly to an amount dependent on the relative motion of the inner and outer tubes.

40. An aesthetic treatment system for treating appearances of a patient's skin associated with a treatment site, comprising:
an aesthetic treatment assembly including an elongate member extending from a handle and configured to be insertable through the skin and a mechanical connective tissue disrupting element attached to a distal end of the elongate member that is capable of expanding at least one region from a smaller state to a wider state and engaging one or more regions of connective tissue subcutaneously, wherein the mechanical connective tissue disrupting element is configurable to both define connective tissue hooking structure as well as connective tissue severing or disrupting structure;
wherein the handle includes an actuating block configured to control operation of the mechanical connective tissue disrupting element, the actuating block including actuating structure that places the mechanical connective tissue disrupting element into connective tissue severing or disrupting structure and facilitates an automatic return of the mechanical connective tissue disrupting element to a home position.

41. The aesthetic treatment system of claim 40, the handle further comprising a follower and a track, wherein the aesthetic treatment system operation is controlled by the follower being guided through the track.

42. A light needle assembly system, comprising:
a handle assembly;
a needle having a terminal end;
a light source configured at the terminal end of the needle, the light source configured to provide transillumination through skin;
a reservoir of anesthesia; and
a spring biased syringe configured to deliver anesthesia through the needle.

43. An assembly for a treatment procedure, comprising:
an elongate assembly including a pushrod extending within the elongate assembly; and
a handle assembly attached to a proximal end of the elongate assembly, the handle assembly including a first part and a second part.

44. The assembly of claim 43, further comprising:
a blade configured at a distal end of the elongate assembly, the blade including teeth that engage openings formed in the elongate assembly; and
wherein manipulating the pushrod causes the blade to deploy from the elongate assembly with the engagement of the teeth and openings controlling movement of the blade.

45. The assembly of claim 43, further comprising:
a hooked blade configured at a distal end of the elongate assembly; and
a pivoting cover configured to cover the bladed hook, the cover operatively associated with the pushrod;
wherein manipulating the pushrod causes the pivoting cover to one or more cover or expose the bladed hook.

45. The assembly of claim 43, further comprising:
a pivoting hooked blade configured at a distal end of the elongate assembly, the pivoting hooked blade being operatively associated with the pushrod; and
a cover configured to cover the bladed hook;
wherein manipulating the pushrod causes the pivoting hooked blade to one or more be covered by the cover or exposed.

47. An assembly for a treatment procedure, comprising:
an elongate assembly including a pushrod extending within the elongate assembly;
a handle assembly attached to a proximal end of the elongate assembly; and
a rotatable blade configured within the elongate assembly and being responsive to manipulation of the handle assembly.

48. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure and includes a flexible band;
wherein the flexible band is configured to block a cutting element of the mechanical septa disrupting element when the mechanical septa disrupting element defines septa hooking structure.

49. An aesthetic treatment system for treating appearances of a patient's skin associated with a treatment site, comprising:
an aesthetic treatment assembly including an elongate member extending from a handle and configured to be insertable through the skin and a mechanical septa disrupting element attached to a distal end of the elongate member that is capable of expanding at least one region from a smaller state to a wider state and engaging one or more regions of septa subcutaneously, wherein the mechanical septa disrupting element is configurable to both define septa hooking structure as well as septa severing or disrupting structure and includes a distal tip;
wherein the tip defines deployable and retractable piercing structure configured for tunneling through tissue.

50. A treatment system for treating appearances of a patient's skin associated with a treatment site, comprising:
an elongate member extending from a handle; and
a septa disrupting element attached to a distal end of the elongate member, wherein the septa disrupting element has a septa hooking position and a septa severing or disrupting position and includes a flexible band, wherein the flexible band blocks a cutting element of the septa disrupting element when the septa disrupting element is in the septa hooking position.
